(19) United States
(12) Patent Application Publication
(10) Pub. No.: US 2004/0259215 A1
(43) Pub. Date: Dec. 23, 2004

(54) Genes coding for dna replication and for proteins related to pathogenesis

(76) Inventors: Oskar Zelder, Speyer (DE); Markus Pompejus, Freinsheim (DE); Hartwig Schroder, Nussloch (DE); Burkhard Kroger, Limburgerhof (DE); Corina Klopprogge, Ludwigshafen (DE); Gregor Haberhauer, Limburgerhof (DE)

Correspondence Address: LAHIVE & COCKFIELD, LLP., 28 STATE STREET, BOSTON, MA, 02109, US

(21) Appl. No.: 10/494,674

(22) PCT Filed: Oct. 31, 2002
(86) PCT No.: PCT/EP02/12132

(30) Foreign Application Priority Data
Nov. 5, 2001 (DE) 101 54 246.1

(51) Int. Cl. C12P 13/04; C12P 13/08; C12N 15/74; C07H 21/04

(52) U.S. Cl. 435/106; 435/252.3; 536/23.2; 435/471; 435/115

(57) ABSTRACT

The invention relates to novel nucleic acid molecules, to the use thereof for constructing genetically improved microorganisms and to methods for preparing fine chemicals, in particular amino acids, with the aid of said genetically improved microorganisms.

### BACKGROUND OF THE INVENTION

Particular products and byproducts of naturally occurring metabolic processes in cells are used in many branches of industry, including the food industry, the animal feed industry, the cosmetics industry and the pharmaceutical industry. These molecules which are collectively referred to as “fine chemicals” comprise organic acids, both proteinogenic and nonproteinogenic amino acids, nucleotides and nucleosides, lipids and fatty acids, diols, carbohydrates, aromatic compounds, vitamins, cofactors and enzymes. They are best produced by means of cultivating, on a large scale, bacteria which have been developed to produce and secrete large amounts of the molecule desired in each particular case. An organism which is particularly suitable for this purpose is Corynebacterium glutamicum, a Gram-positive nonpathogenic bacterium. Using strain selection, a number of mutant strains have been developed which produce various desirable compounds. The selection of strains which are improved with respect to the production of a particular molecule is, however, a time-consuming and difficult process.

### BRIEF DESCRIPTION OF THE INVENTION

The present invention provides novel nucleic acid molecules which can be used for identifying or classifying Corynebacterium glutamicum or related bacterial species. C. glutamicum is a Gram-positive, aerobic bacterium which is widely used in industry for the large-scale production of a number of fine chemicals and also for the degradation of hydrocarbons (for example in the case of crude oil spills) and for the oxidation of terpenoids. The nucleic acid molecules may therefore be used for identifying microorganisms which can be used for producing fine chemicals, for example by fermentation processes. Although C. glutamicum itself is nonpathogenic, it is, however, related to other Corynebacterium species such as Corynebacterium diphteriae (the diphtheria pathogen), which are major pathogens in humans. The ability to identify the presence of Corynebacterium species may therefore also be of significant clinical importance, for example in diagnostic applications. Moreover, said nucleic acid molecules may serve as reference points for mapping the C. glutamicum genome or genomes of related organisms.

These novel nucleic acid molecules encode proteins which are referred to herein as DNA-replication, ribosomal and pathogenesis (RRP) proteins. These RRP proteins, for example, may be involved directly or indirectly in the production of one or more fine chemicals in C. glutamicum. The RRP proteins of the invention may also be involved in the degradation of hydrocarbons or in the oxidation of terpenoids. These proteins can be used for identifying Corynebacterium glutamicum or organisms related to C. glutamicum; the presence of an RRP protein specific for C. glutamicum and related species in a protein mixture may indicate the presence of any of said bacteria in the sample. Furthermore, said RRP proteins may have homologs in plants or animals, which are involved in a diseased state or an illness; thus, said proteins may serve as useful pharmaceutical targets for drug screening and for the development of therapeutic compounds.

Owing to the availability of cloning vectors for use in Corynebacterium glutamicum, as disclosed, for example in Sinskey et al., U.S. Pat. No. 4,649,119, and of techniques for the genetic manipulation of C. glutamicum and the related Brevibacterium species (e.g. lactofermentum) (Yoshihama et al., J. Bacteriol. 162: 591-597 (1985); Katsumata et al., J. Bacteriol. 159: 306-311 (1984); and Santamaria et al. J. Gen. Microbiol. 130: 2237-2246 (1984)), the nucleic acid molecules of the invention can be used for genetic manipulation of said organism in order to modulate the production of one or more fine chemicals. This modulation may take place due to a direct effect of the manipulation of a gene of the invention or due to an indirect effect of such a manipulation. For example, it is possible, by modifying the activity of a protein which is involved in the biosynthesis or degradation of a fine chemical (i.e. by mutagenesis of the corresponding gene), to directly modulate the ability of the cell to synthesize or degrade said compound, thereby modulating the yield and/or efficiency of production of said fine chemical. Likewise, it is possible, by modulating the activity of a protein which regulates a metabolic pathway of a fine chemical, to have a direct influence on whether production of the compound of interest is up- or downregulated, with both of these processes modulating the yield or efficiency of production of the fine chemical from the cell.

Modification of the activity of a protein of the invention (i.e. mutagenesis of the corresponding gene) may also modulate indirectly the production of fine chemicals so that the ability of the cell to grow and to divide or to remain viable and productive is increased overall. Fine chemicals are usually produced from C. glutamicum by a large-scale fermentative culture of these microorganisms under conditions which frequently are suboptimal for growth and cell division. It may be possible, by modifying a protein of the invention (e.g. a stress reaction protein, a cell wall protein or a protein involved in the metabolism of compounds required for cell division and cell growth to take place, such as nucleotides and amino acids) so that better survival, growth and propagation is possible under said conditions, to increase the number and productivity of these modified C. glutamicum cells in large-scale cultures, and this should in turn lead to increased yields and/or increased efficiency of production of one or more fine chemicals of interest. Furthermore, the metabolic pathways of a cell are necessarily dependent on one another and coregulated. It is possible, by changing the activity of any metabolic pathway in C. glutamicum (i.e. by changing the activity of any of the proteins of the invention, which are involved in such a pathway), to change simultaneously the activity or regulation of another metabolic pathway in said microorganism, which may be involved directly in the synthesis or degradation of a fine chemical.

There is a number of mechanisms by which modification of an RRP protein of the invention influences the yield, production or productivity of a fine chemical in a C. glutamicum strain containing such a modified RRP protein. For example, it is possible, by accelerating the rate of DNA replication (for example by optimizing the activity of one or more DNA polymerases or by increasing the rate at which a topoisomerase or helicase unwinds the DNA), to accelerate cell division, and this in turn increases the number of living fine chemical-producing cells in a culture. In a similar way, it is possible, by increasing the rate of translation (e.g. by optimizing the activity of ribosomal proteins), to increase the number of proteins in a cell, which are involved in the synthesis of one or more fine chemicals of interest. Both measures should lead to an increase in the production of the fine chemical of interest when culturing microorganisms having appropriately modified RRP proteins.

Modifications in the DNA replication proteins of the invention may also lead to greater replication accuracy and thereby increase the genetic stability and viability of said microorganisms and thereby reduce the risk of destroying a further genetic modification which increases production of said fine chemical, due to incorrect replication.

The present invention provides novel nucleic acid molecules encoding proteins which are referred to herein as RRP proteins and which, for example, can modulate the production or efficiency of production of one or more fine chemicals in C. glutamicum or serve as identification markers for C. glutamicum or related organisms. Nucleic acid molecules encoding an RRP protein are referred to herein as RRP nucleic acid molecules. In a preferred embodiment, the RRP protein can modulate the production or efficiency of production of one or more fine chemicals in C. glutamicum or serve as identification marker for C. glutamicum or related organisms. Examples of such proteins are those encoded by the genes listed in Table 1.

Consequently, one aspect of the invention relates to isolated nucleic acid molecules (e.g. cDNAs) comprising a nucleotide sequence which encodes an RRP protein or biologically active sections thereof and also nucleic acid fragments which are suitable as primers or hybridization probes for detecting or amplifying RRP-encoding nucleic acid (e.g. DNA or mRNA). In particularly preferred embodiments, the isolated nucleic acid molecule comprises any of the nucleotide sequences listed in Appendix A or the coding region or a complement thereof of any of these nucleotide sequences. In other preferred embodiments, the isolated nucleic acid molecule encodes any of the amino acid sequences listed in Appendix B. The preferred RRP proteins of the invention likewise have preferably at least one of the RRP activities described herein.

Appendix A defines hereinbelow the nucleic acid sequences of the sequence listing together with the sequence modifications at the relevant position, described in Table 1.

Appendix B defines hereinbelow the polypeptide sequences of the sequence listing together with the sequence modifications at the relevant position, described in Table 1.

In a further embodiment, the isolated nucleic acid molecule is at least 15 nucleotides in length and hybridizes under stringent conditions to a nucleic acid molecule which comprises a nucleotide sequence of Appendix A. The isolated nucleic acid molecule preferably corresponds to a naturally occurring nucleic acid molecule. The isolated nucleic acid more preferably encodes a naturally occurring C. glutamicum RRP protein or a biologically active section thereof.

A further aspect of the invention relates to vectors, for example recombinant expression vectors, which contain the nucleic acid molecules of the invention and to host cells into which said vectors have been introduced. In one embodiment, an RRP protein is prepared by using this host cell which is cultivated in a suitable medium. The RRP protein may then be isolated from the medium or the host cell.

A further aspect of the invention relates to a genetically modified microorganism into which an RRP gene has been introduced or in which an RRP gene has been modified. In one embodiment, the genome of said microorganism has been modified by introducing at least one inventive nucleic acid molecule which encodes the mutated RRP sequence as transgene. In another embodiment, an endogenous RRP gene in the genome of said microorganism has been modified, for example, functionally disrupted, by homologous recombination with a modified RRP gene. In a preferred embodiment, the microorganism belongs to the genus Corynebacterium or Brevibacterium, with Corynebacterium glutamicum being particularly preferred. In a preferred embodiment, the microorganism is also used for preparing a compound of interest, such as an amino acid, particularly preferably lysine.

Another preferred embodiment are host cells having more than one of the nucleic acid molecules described in Appendix A. Such host cells can be prepared in various ways known to the skilled worker. They may be transfected, for example, by vectors carrying several of the nucleic acid molecules of the invention. However, it is also possible to use a vector for introducing in each case one nucleic acid molecule of the invention into the host cell and therefore to use a plurality of vectors either simultaneously or sequentially. Thus it is possible to construct host cells which carry numerous, up to several hundred, nucleic acid sequences of the invention. Such an accumulation can often produce superadditive effects on the host cell with respect to fine-chemical productivity.

A further aspect of the invention relates to an isolated RRP protein or a section thereof, for example a biologically active section. In a preferred embodiment, the isolated RRP protein or its section may modulate the production or efficiency of production of one or more fine chemicals in C. glutamicum or serve as identification marker for C. glutamicum or related organisms. In another preferred embodiment, the isolated RRP protein or a section thereof is sufficiently homologous to an amino acid sequence of Appendix B so that the protein or its section retains the ability, for example, to modulate the production or efficiency of production of one or more fine chemicals in C. glutamicum or to serve as identification marker for C. glutamicum or related organisms.

Moreover, the invention relates to an isolated RRP protein preparation. In preferred embodiments, the RRP protein comprises an amino acid sequence of Appendix B. In a further preferred embodiment, the invention relates to an isolated full-length protein which is essentially homologous to a complete amino acid sequence of Appendix B (which is encoded by an open reading frame in Appendix A).

The RRP polypeptide or a biologically active section thereof may be functionally linked to a non-RRP polypeptide in order to produce a fusion protein. In preferred embodiments, this fusion protein has a different activity from that of the RRP protein alone. In other preferred embodiments, said fusion protein can modulate the yield, production and/or efficiency of production of one or more fine chemicals in C. glutamicum or serve as identification marker for C. glutamicum or related organisms. In particularly preferred embodiments, integration of said fusion protein into a host cell modulates the production of a compound of interest by the cell.

A further aspect of the invention relates to a method for preparing a fine chemical. The method provides for the cultivation of a cell containing a vector which causes expression of an RRP nucleic acid molecule of the invention so that a fine chemical is produced. In a preferred embodiment, this method moreover comprises the step of obtaining a cell containing such a vector, said cell being transfected with a vector which causes expression of an RRP nucleic acid. In a further preferred embodiment, said method moreover comprises the step in which the fine chemical is obtained from the culture. In a preferred embodiment, the cell belongs to the genus Corynebacterium or Brevibacterium.

A further aspect of the invention relates to methods for modulating the production of a molecule from a microorganism. These methods comprise contacting the cell with a substance which modulates RRP-protein activity or RRP nucleic-acid expression such that a cell-associated activity is modified in comparison with the same activity in the absence of said substance. In a preferred embodiment, the cell is modulated with regard to one or more C. glutamicum RRP protein activities so as to improve the yield, production and/or efficiency of production of a fine chemical of interest by this microorganism. The substance which modulates RRP protein activity can be a substance which stimulates, for example, RRP-protein activity or RRP nucleic-acid expression. Examples of substances stimulating RRP protein activity or RRP nucleic-acid expression include small molecules, active RRP proteins and nucleic acids which encode RRP proteins and have been introduced into the cell. Examples of substances which inhibit RRP activity or RRP expression include small molecules and RRP antisense nucleic acid molecules.

A further aspect of the invention relates to methods for modulating the yields, the production and/or the efficiency of production of a compound of interest from a cell, comprising introducing an RRP wild-type gene or RRP-mutant gene into a cell, which gene either remains on a separate plasmid or is integrated into the genome of the host cell. Integration into the genome may take place randomly or via homologous recombination so that the native gene is replaced by the integrated copy, leading to the production of the compound of interest from the cell to be modulated. In a preferred embodiment, said yields are increased. In a further preferred embodiment, the chemical is a fine chemical which, in a particularly preferred embodiment, is an amino acid. In a particularly preferred embodiment, this amino acid is L-lysine.

### DETAILED DESCRIPTION OF THE INVENTION

The present invention provides RRP-nucleic acid and RRP-protein molecules which may be used for identifying Corynebacterium glutamicum or related organisms, for mapping the C. glutamicum genome (or the genome of a closely related organism) or for identifying microorganisms which may be used for producing fine chemicals, for example by fermentative processes. The proteins encoded by said nucleic acids may be used for directly or indirectly modulating the production or efficiency of production of one or more fine chemicals in C. glutamicum, as identification markers for C. glutamicum or related organisms, for oxidizing terpenoids or for degrading hydrocarbons or as targets for developing therapeutic pharmaceutical compounds. The aspects of the invention are further illustrated below.

I. Fine Chemicals

The term “fine chemicals” is known in the art and includes molecules which are produced by an organism and are used in various branches of industry such as, for example, but not restricted to, the pharmaceutical industry, the agricultural industry and the cosmetics industry. These compounds comprise organic acids such as tartaric acid, itaconic acid and diaminopimelic acid, both proteinogenic and nonproteinogenic amino acids, purine and pyrimidine bases, nucleosides and nucleotides (as described, for example, in Kuninaka, A. (1996) Nucleotides and related compounds, pp. 561-612, in Biotechnology Vol. 6, Rehm et al., Editors VCH: Weinheim and the references therein), lipids, saturated and unsaturated fatty acids (e.g. arachidonic acid), diols (e.g. propanediol and butanediol), carbohydrates (e.g. hyaluronic acid and trehalose), aromatic compounds (e.g. aromatic amines, vanilline and indigo), vitamins and cofactors (as described in Ullmann's Encyclopedia of Industrial Chemistry, Vol. A27, “Vitamins”, pp. 443-613(1996) VCH: Weinheim and the references therein; and Ong, A. S., Niki, E. and Packer, L. (1995) “Nutrition, Lipids, Health and Disease” Proceedings of the UNESCO/Confederation of Scientific and Technological Associations in Malaysia and the Society for Free Radical Research—Asia, held Sep. 1-3, 1994 in Penang, Malaysia, AOCS Press (.1995)), enzymes and all other chemicals described by Gutcho (1983) in Chemicals by Fermentation, Noyes Data Corporation, ISBN: 0818805086 and the references indicated therein. The metabolism and the uses of particular fine chemicals are further illustrated below.

A. Amino Acid Metabolism and Uses

Amino acids comprise the fundamental structural units of all proteins and are thus essential for normal functions in all organisms. The term “amino acid” is known in the art. Proteinogenic amino acids, of which there are 20 types, serve as structural units for proteins, in which they are linked together by peptide bonds, whereas the nonproteinogenic amino acids (hundreds of which are known) usually do not occur in proteins (see Ullmann's Encyclopedia of Industrial Chemistry, Vol. A2, pp. 57-97 VCH: Weinheim (1985)). Amino acids can exist in the optical D or L configuration, although L-amino acids are usually the only type found in naturally occurring proteins. Biosynthetic and degradation pathways of each of the 20 proteinogenic amino acids are well characterized both in prokaryotic and eukaryotic cells (see, for example, Stryer, L., Biochemistry, 3^{rd} edition, pp. 578-590 (1988)). The “essential” amino acids (histidine, isoleucine, leucine, lysine, methionine, phenylalanine, threonine, tryptophan and valine), so called because, owing to the complexity of their biosyntheses, they must usually be taken in with the diet, are converted by simple biosynthetic pathways into the other 11 “nonessential” amino acids (alanine, arginine, asparagine, aspartate, cysteine, glutamate, glutamine, glycine, proline, serine and tyrosine). Higher animals are able to synthesize some of these amino acids but the essential amino acids must be taken in with the food in order that normal protein synthesis takes place.

Apart from their function in protein biosynthesis, these amino acids are interesting chemicals as such, and it has been found that many have various applications in the human food, animal feed, chemicals, cosmetics, agricultural and pharmaceutical industries. Lysine is an important amino acid not only for human nutrition but also for monogastric livestock such as poultry and pigs. Glutamate is most frequently used as flavor additive (monosodium glutamate, MSG) and elsewhere in the food industry, as are aspartate, phenylalanine, glycine and cysteine. Glycine, L-methionine and tryptophan are all used in the pharmaceutical industry. Glutamine, valine, leucine, isoleucine, histidine, arginine, proline, serine and alanine are used in the pharmaceutical industry and the cosmetics industry. Threonine, tryptophan and D/L-methionine are widely used animal feed additives (Leuchtenberger, W. (1996) Amino acids—technical production and use, pp. 466-502 in Rehm et al., (editors) Biotechnology Vol. 6, Chapter 14a, VCH: Weinheim). It has been found that these amino acids are additionally suitable as precursors for synthesizing synthetic amino acids and proteins, such as N-acetylcysteine, S-carboxymethyl-L-cysteine, (S)-5-hydroxytryptophan and other substances described in Ullmann's Encyclopedia of Industrial Chemistry, Vol. A2, pp. 57-97, VCH, Weinheim, 1985.

The biosynthesis of these natural amino acids in organisms able to produce them, for example bacteria, has been well characterized (for a review of bacterial amino acid biosynthesis and its regulation see Umbarger, H. E. (1978) Ann. Rev. Biochem. 47: 533-606). Glutamate is synthesized by reductive amination of α-ketoglutarate, an intermediate product in the citric acid cycle. Glutamine, proline and arginine are each generated successively from glutamate. The biosynthesis of serine takes place in a three-step process, starts with 3-phosphoglycerate (an intermediate product of glycolysis) and affords this amino acid after oxidation, transamination and hydrolysis steps. Cysteine and glycine are each produced from serine, specifically the former by condensation of homocysteine with serine, and the latter by transfer of the side-chain β-carbon atom to tetrahydrofolate in a reaction catalyzed by serine transhydroxy-methylase. Phenylalanine and tyrosine are synthesized from the precursors of the glycolysis and pentose phosphate pathway, and erythrose 4-phosphate and phosphoenolpyruvate, in a 9-step biosynthetic pathway which diverges only in the last two steps after the synthesis of prephenate. Tryptophan is likewise produced from these two starting molecules but it is synthesized by an 11-step pathway. Tyrosine can also be prepared from phenylalanine in a reaction catalyzed by phenylalanine hydroxylase. Alanine, valine and leucine are each biosynthetic products derived from pyruvate, the final product of glycolysis. Aspartate is formed from oxalacetate, an intermediate product of the citrate cycle. Asparagine, methionine, threonine and lysine are each produced by the conversion of aspartate. Isoleucine is formed from threonine. Histidine is formed from 5-phosphoribosyl 1-pyrophosphate, an activated sugar, in a complex 9-step pathway.

Amounts of amino acids exceeding those required for protein biosynthesis cannot be stored and are instead broken down so that intermediate products are provided for the principal metabolic pathways in the cell (for a review, see Stryer, L., Biochemistry, 3^{rd} edition, Chapter 21 “Amino Acid Degradation and the Urea Cycle”; pp. 495-516 (1988)). Although the cell is able to convert unwanted amino acids into the useful intermediate products of metabolism, production of amino acids is costly in terms of energy, the precursor molecules and the enzymes necessary for their synthesis. It is therefore not surprising that amino acid biosynthesis is regulated by feedback inhibition, whereby the presence of a particular amino acid slows down or completely stops its own production (for a review of feedback mechanisms in amino acid biosynthetic pathways, see Stryer, L., Biochemistry, 3^{rd} edition, Chapter 24, “Biosynthesis of Amino Acids and Heme”, pp. 575-600 (1988)). The output of a particular amino acid is therefore restricted by the amount of this amino acid in the cell.

B. Metabolism and Uses of Vitamins, Cofactors and Nutraceuticals

Vitamins, cofactors and nutraceuticals comprise another group of molecules. Higher animals have lost the ability to synthesize them and therefore have to take them in, although they are easily synthesized by other organisms such as bacteria. These molecules are either bioactive molecules per se or precursors of bioactive substances which serve as electron transfer molecules or intermediate products in a number of metabolic pathways. Besides their nutritional value, these compounds also have a significant industrial value as colorants, antioxidants and catalysts or other processing auxiliaries. (For a review of the structure, activity and industrial applications of these compounds, see, for example, Ullmann's Encyclopedia of Industrial Chemistry, “Vitamins”, Vol. A27, pp. 443-613, VCH: Weinheim, 1996). The term “vitamin” is known in the art and comprises nutrients which are required for normal functional of an organism but cannot be synthesized by this organism itself. The group of vitamins may include cofactors and nutraceutical compounds. The term “cofactor” comprises nonproteinaceous compounds necessary for the appearance of a normal enzymic activity. These compounds may be organic or inorganic; the cofactor molecules of the invention are preferably organic. The term “nutraceutical” comprises food additives which are health-promoting in plants and animals, especially humans. Examples of such molecules are vitamins, antioxidants and likewise certain lipids (e.g. polyunsaturated fatty acids).

The biosynthesis of these molecules in organisms able to produce them, such as bacteria, has been comprehensively characterized (Ullmann's Encyclopedia of Industrial Chemistry, “Vitamins”, Vol. A27, pp. 443-613, VCH: Weinheim, 1996, Michal, G. (1999) Biochemical Pathways: An Atlas of Biochemistry and Molecular Biology, John Wiley & Sons; Ong, A. S., Niki, E. and Packer, L. (1995) “Nutrition, Lipids, Health and Disease” Proceedings of the UNESCO/Confederation of Scientific and Technological Associations in Malaysia and the Society for free Radical Research—Asia, held on Sep. 1-3, 1994, in Penang, Malaysia, AOCS Press, Champaign, IL X, 374 S).

Thiamine (vitamin B₁) is formed by chemical coupling of pyrimidine and thiazole units. Riboflavin (vitamin B₂) is synthesized from guanosine 5′-triphosphate (GTP) and ribose 5′-phosphate. Riboflavin in turn is employed for the synthesis of flavin mononucleotide (FMN) and flavin adenine dinucleotide (FAD). The family of compounds together referred to as “vitamin B6” (for example pyridoxine, pyridoxamine, pyridoxal 5′-phosphate and the commercially used pyridoxine hydrochloride), are all derivatives of the common structural unit 5-hydroxy-6-methylpyridine. Panthothenate (pantothenic acid, R-(+)-N-(2,4-dihydroxy-3,3-dimethyl-1-oxobutyl)-β-alanine) can be prepared either by chemical synthesis or by fermentation. The last steps in pantothenate biosynthesis consist of ATP-driven condensation of β-alanine and pantoic acid. The enzymes responsible for the biosynthetic steps for the conversion into pantoic acid and into β-alanine and for the condensation to pantothenic acid are known. The metabolically active form of pantothenate is coenzyme A whose biosynthesis takes place by 5 enzymatic steps. Pantothenate, pyridoxal 5′-phosphate, cysteine and ATP are the precursors of coenzyme A. These enzymes catalyze not only the formation of pantothenate but also the production of (R)-pantoic acid, (R)-pantolactone, (R)-panthenol (provitamin B₅), pantetheine (and its derivatives) and coenzyme A.

The biosynthesis of biotin from the precursor molecule pimeloyl-CoA in microorganisms has been investigated in detail, and several of the genes involved have been identified. It has emerged that many of the corresponding proteins are involved in the Fe cluster synthesis and belong to the class of nifS proteins. Liponic acid is derived from octanoic acid and serves as coenzyme in energy metabolism where it is a constituent of the pyruvate dehydrogenase complex and of the α-ketoglutarate dehydrogenase complex. Folates are a group of substances all derived from folic acid which in turn is derived from L-glutamic acid, p-aminobenzoic acid and 6-methylpterin. The biosynthesis of folic acid and its derivatives starting from the intermediate products of the biotransformation of guanosine 5′-triphosphate (GTP), L-glutamic acid and p-aminobenzoic acid has been investigated in detail in certain microorganisms.

Corrinoids (such as the cobalamines and, in particular, vitamin B₁₂) and the porphyrins belong to a group of chemicals distinguished by a tetrapyrrole ring system. The biosynthesis of vitamin B₁₂ is so complex that it has not yet been completely characterized, but many of the enzymes and substrates involved are now known. Nicotinic acid (nicotinate) and nicotinamide are pyridine derivatives which are also referred to as “niacin”. Niacin is the precursor of the important coenzymes NAD (nicotinamide adenine dinucleotide) and NADP (nicotinamide adenine dinucleotide phosphate) and their reduced forms.

Production of these compounds on the industrial scale is mostly based on cell-free chemical syntheses, although some of these chemicals, such as riboflavin, vitamin B₆, pantothenate and biotin, have also been produced by large-scale cultivation of microorganisms. Only vitamin B₁₂ is, because of the complexity of its synthesis, produced only by fermentation. In vitro processes require a considerable expenditure of materials and time and frequently high costs.

C. Purine, Pyrimidine, Nucleoside and Nucleotide Metabolism and Uses

Genes for purine and pyrimidine metabolism and their corresponding proteins are important aims for the therapy of oncoses and viral infections. The term “purine” or “pyrimidine” comprises nitrogen-containing bases which form part of nucleic acids, coenzymes and nucleotides. The term “nucleotide” encompasses the fundamental structural units of nucleic acid molecules, which comprise a nitrogen-containing base, a pentose sugar (the sugar is ribose in the case of RNA and the sugar is D-deoxyribose in the case of DNA) and phosphoric acid. The term “nucleoside” comprises molecules which serve as precursors of nucleotides but have, in contrast to the nucleotides, no phosphoric acid unit. It is possible to inhibit RNA and DNA synthesis by inhibiting the biosynthesis of these molecules or their mobilization to form nucleic acid molecules; targeted inhibition of this activity in cancer cells allows the ability of tumor cells to divide and replicate to be inhibited. There are also nucleotides which do not form nucleic acid molecules but serve as energy stores (i.e. AMP) or as coenzymes (i.e. FAD and NAD).

Several publications have described the use of these chemicals for these medical indications, the purine and/or pyrimidine metabolism being influenced (for example Christopherson, R. I. and Lyons, S. D. (1990) “Potent inhibitors of de novo pyrimidine and purine biosynthesis as chemotherapeutic agents”, Med. Res. Reviews 10: 505-548). Investigations of enzymes involved in purine and pyrimidine metabolism have concentrated on the development of novel medicaments which can be used, for example, as immunosuppressants or antiproliferative agents (Smith, J. L. (1995) “Enzymes in Nucleotide Synthesis” Curr. Opin. Struct. Biol. 5: 752-757; Simmonds, H. A. (1995) Biochem. Soc. Transact. 23: 877-902). However, purine and pyrimidine bases, nucleosides and nucleotides also have other possible uses: as intermediate products in the biosynthesis of various fine chemicals (e.g. thiamine, S-adenosylmethionine, folates or riboflavin), as energy carriers for the cell (for example ATP or GTP) and for chemicals themselves, which are ordinarily used as flavor enhancers (for example IMP or GMP) or for many medical applications (see, for example, Kuninaka, A., (1996) “Nucleotides and Related Compounds in Biotechnology” Vol. 6, Rehm et al., editors VCH: Weinheim, pp. 561-612). Enzymes involved in purine, pyrimidine, nucleoside or nucleotide metabolism are also increasingly serving as targets against which chemicals are being developed for crop protection, including fungicides, herbicides and insecticides.

The metabolism of these compounds in bacteria has been characterized (for reviews, see, for example, Zalkin, H. and Dixon, J. E. (1992) “De novo purine nucleotide biosynthesis” in Progress in Nucleic Acids Research and Molecular biology, Vol. 42, Academic Press, pp. 259-287; and Michal, G. (1999) “Nucleotides and Nucleosides”; Chapter 8 in : Biochemical Pathways: An Atlas of Biochemistry and Molecular Biology, Wiley, N.Y.). Purine metabolism, the object of intensive research, is essential for normal functioning of the cell. Disordered purine metabolism in higher animals may cause severe illnesses, for example gout. Purine nucleotides are synthesized from ribose 5-phosphate by a number of steps via the intermediate compound inosine 5′-phosphate (IMP), leading to the production of guanosine 5′-monophosphate (GMP) or adenosine 5′-monophosphate (AMP), from which the triphosphate forms used as nucleotides can easily be prepared. These compounds are also used as energy stores, so that breakdown thereof provides energy for many different biochemical processes in the cell. Pyrimidine biosynthesis takes place via formation of uridine 5′-mono-phosphate (UMP) from ribose 5-phosphate. UMP in turn is converted into cytidine 5′-triphosphate (CTP). The deoxy forms of all nucleotides are prepared in a one-step reduction reaction from the diphosphate ribose form of the nucleotide to give the diphosphate deoxyribose form of the nucleotide. After phosphorylation, these molecules can take part in DNA synthesis.

D. Trehalose Metabolism and Uses

Trehalose consists of two glucose molecules linked together by an α,α-1,1 linkage. It is ordinarily used in the food industry as sweetener, as additive for dried or frozen foods and in beverages. However, it is also used in the pharmaceutical industry or in the cosmetics industry and biotechnology industry (see, for example, Nishimoto et al., (1998) U.S. Pat. No. 5,759,610; Singer, M. A. and Lindquist, S. (1998) Trends Biotech. 16: 460-467; Paiva, C. L. A. and Panek, A. D. (1996) Biotech Ann. Rev. 2: 293-314; and Shiosaka, M. (1997) J. Japan 172: 97-102). Trehalose is produced by enzymes of many microorganisms and is naturally released into the surrounding medium from which it can be isolated by methods known in the art.

II. Activity of the Genes of the Invention

At least three activities are required for a particular bacterial species to survive in an environment. Firstly, the cell must be able to divide efficiently for at least to maintain or to increase the cell population. Secondly, the cell must efficiently express those genes which encode proteins required for normal cellular functions. Finally, the cell must be capable of interacting with its environment, either by adapting to the prevailing environmental conditions or by physical locomotion in preferred environments or by acting directly on the environment in a manner so as to improve its survivability. The critical steps involved in any of these activities include replication of the bacterial genome, ribosomal activity in protein biosynthesis and anticellular or cell lysis activities (as they are involved in the pathogenesis of an organism).

III. Elements and Methods of the Invention

The present invention is based, at least partially, on the detection of new molecules which are referred to herein as RRP nucleic-acid molecules. These RRP nucleic acid molecules are suitable not only for identifying C. glutamicum or related bacterial species but also as markers for mapping the C. glutamicum genome and for identifying bacteria suitable for producing fine chemicals by, for example, fermentative processes. The present invention is based, at least partially, also on those RRP-protein molecules which are encoded by said RRP nucleic acid molecules. These RRP molecules may modulate the yield, production and/or efficiency of production of one or more fine chemicals in C. glutamicum, serve as identification markers for C. glutamicum or related organisms, degrade hydrocarbons and serve as targets for the development of therapeutic pharmaceutical compounds. In one embodiment, the RRP molecules of the invention are directly or indirectly involved in the metabolic pathway of one or more fine chemicals in C. glutamicum. In a preferred embodiment, the activity of the inventive RRP molecules of taking part indirectly or directly in such metabolic pathways affects the production of a fine chemical of interest by this microorganism. In a particularly preferred embodiment, the activity of the RRP molecules of the invention is modulated such that the C. glutamicum metabolic pathways in which the RRP proteins of the invention are involved are modulated with regard to efficiency or output, and this modulates directly or indirectly the production or efficiency of production of a fine chemical of interest by C. glutamicum.

The term “RRP protein” or “RRP polypeptide” comprises proteins whihc are capable of modulating the yield, production and/or efficiency of production of one or more fine chemicals in C. glutamicum, degrading hydrocarbons, oxidizing terpenoids, and serving as target protein for drug screening or drug design or as identification markers for C. glutamicum or related organisms. Examples of RRP proteins comprise those which are encoded by the RRP genes listed in Table 1 and Appendix A. The terms “RRP gene” and “RRP nucleic acid sequence” comprise nucleic acid sequences encoding an RRP protein which comprises a coding region and corresponding untranslated 5′ and 3′ sequence regions. Examples of RRP genes are the ones listed in Table 1. The terms “production” and “productivity” are known in the art and include the concentration of the fermentation products (for example of the fine chemical of interest, which is produced within a predetermined time interval and a predetermined fermentation volume (e.g. kg of product per h per 1)). The term “efficiency of production” comprises the time required by the cell for reaching a particular production quantity (for example, the time required by the cell for reaching a particular output rate of a fine chemical). The term “yield” or “product/carbon yield” is known in the art and comprises the efficiency of converting the carbon source into the product (i.e. the fine chemical). This is, for example, usually expressed as kg of product per kg of carbon source. Increasing the yield or production of the compound increases the amount of the molecules obtained or of the suitable obtained molecules of this compound in a particular culture volume over a predetermined period. The terms “biosynthesis” and “biosynthetic pathway” are known in the art and comprise the synthesis of a compound, preferably an organic compound, from intermediates by a cell, for example in a multistep process or highly regulated process. The terms “degradation” and “degradation pathway” are known in the art and comprise cleavage of a compound, preferably an organic compound, into degradation products (in more general terms: smaller or less complex molecules) by a cell, for example in a multistep process or highly regulated process. The term “metabolism” is known in the art and comprises the entirety of biochemical reactions which take place in an organism. The metabolism of a particular compound (e.g. the metabolism of an amino acid such as glycine) then comprises all biosynthetic, modification and degradation pathways in the cell which affect this compound.

In another embodiment, the RRP molecules of the invention are capable of modulating directly or indirectly the production of a molecule of interest, such as a fine chemical, in a microorganism such as C. glutamicum. Using gene recombination techniques, it is possible to manipulate one or more RRP proteins of the invention so that their function is modulated. This modulation of the function may lead to modulation of the yield, production and/or efficiency of production of one or more fine chemicals from C. glutamicum.

For example, it is possible, by modifying the activity of a protein involved in the biosynthesis or degradation of a fine chemical (i.e. by mutagenesis of the corresponding gene), to directly modulate the ability of the cell to synthesize or degrade this compound and thereby to modulate the yield and/or efficiency of production of the fine chemicals. Likewise, it is possible, by modulating the activity of a protein regulating a fine-chemical metabolic pathway, to have a direct influence on whether the production of the compound of interest is up- or downregulated, with both processes modulating the yield or efficiency of production of said fine chemical from the cell.

The production of fine chemicals may also be modulated indirectly by modifying the activity of a protein of the invention (i.e. by mutating the corresponding gene) so that the ability of the cell to grow and to divide ought to remain viable and productive is increased overall. Fine chemicals are usually produced from C. glutamicum by large-scale fermentative culture of these microorganisms under conditions which frequently are suboptimal for growth and cell division. By modifying a protein of the invention (for example, a stress reaction protein, a cell wall protein or proteins involved in the metabolism of compounds which are required for cell growth and cell division to take place, such as nucleotides and amino acids) so as to be able to improve the survival, growth and propagation in these conditions, it may be possible to increase the number and productivity of these modified C. glutamicum cells in large-scale cultures, and this should in turn lead to increased yields and/or increased efficiency of production of one or more fine chemicals of interest. Furthermore, the metabolic pathways of a cell are necessarily dependent on one another and coregulated. Changing the activity of any metabolic pathway in C. glutamicum (i.e. changing the activity of any of the proteins of the invention, which is involved in such a pathway) makes it possible to change in this microorganism simultaneously the activity or regulation of another metabolic pathway which may be involved directly in the synthesis or degradation of a fine chemical.

The isolated nucleic acid sequences of the invention are located in the genome of a Corynebacterium glutamicum strain which can be obtained from the American Type Culture Collection under the name ATCC 13032. The nucleotide sequence of the isolated C. glutamicum RRP nucleic acid molecules and the predicted amino acid sequences of the C. glutamicum RRP proteins are shown in Appendix A and B, respectively. Computer analyses were carried out, which classified and/or identified many of these nucleotide sequences as sequences with homology to E. coli genes or Bacillus subtilis genes.

The present invention also relates to proteins whose amino acid sequence is essentially homologous to an amino acid sequence in Appendix B. As used herein, a protein whose amino acid sequence is essentially homologous to a selected amino acid sequence is at least about 50% homologous to the selected amino acid sequence, for example to the entire selected amino acid sequence. A protein whose amino acid sequence is essentially homologous to a selected amino acid sequence may also be at least about 50-60%, preferably at least about 60-70%, more preferably at least about 70-80%, 80-90% or 90-95%, and most preferably at least about 96%, 97%, 98%, 99%, homologous or even more homologous to the selected amino acid sequence.

An RRP protein of the invention or a biologically active section or fragment thereof can modulate the yield, production and/or efficiency of production of one or more fine chemicals in C. glutamicum, degrade hydrocarbons, oxidize terpenoids, serve as a target for drug development or serve as identification marker for C. glutamicum or related organisms.

The following subsections describe various aspects of the invention in more detail:

A. Isolated Nucleic Acid Molecules

One aspect of the invention relates to isolated nucleic acid molecules which encode RRP polypeptides or biologically active sections thereof and to nucleic acid fragments which are sufficient for the use as hybridization probes or primers for identifying or amplifying RRP-encoding nucleic acids (e.g. RRP DNA). These nucleic acid molecules may be used for identifying C. glutamicum or related organisms, for mapping the genome of C. glutamicum or of related organisms or for identifying microorganisms which are suitable for producing fine chemicals, for example by fermentative processes. The term “nucleic acid molecule”, as used herein, is intended to comprise DNA molecules (e.g. cDNA or genomic DNA) and RNA molecules (e.g. mRNA) and also DNA or RNA analogs generated by means of nucleotide analogs. Moreover, this term comprises the untranslated sequence located at the 3′ and 5′ ends of the coding gene region: at least about 100 nucleotides of the sequence upstream of the 5′ end of the coding region and at least about 20 nucleotides of the sequence downstream of the 3′ end of the coding region of the gene. The nucleic acid molecule may be single-stranded or double-stranded but is preferably a double-stranded DNA. An “isolated” nucleic acid molecule is removed from other nucleic acid molecules which are present in the natural source of the nucleic acid. An “isolated” nucleic acid preferably does not have any sequences which flank the nucleic acid naturally in the genomic DNA of the organism from which the nucleic acid originates (for example sequences located at the 5′ or 3′ end of the nucleic acid). In various embodiments, the isolated RRP nucleic acid molecule may have, for example, less than about 5 kb, 4 kb, 3 kb, 2 kb, 1 kb, 0.5 kb or 0.1 kb of the nucleotide sequences which naturally flank the nucleic acid molecule in the genomic DNA of the cell from which the nucleic acid originates (e.g. a C. glutamicum cell). In addition to this, an “isolated” nucleic acid molecule such as a cDNA molecule may be essentially free of another cellular material or culture medium, if prepared by recombinant techniques, or free of chemical precursors or other chemicals, if synthesized chemically.

A nucleic acid molecule of the invention, for example a nucleic acid molecule having a nucleotide sequence of Appendix A or a section thereof, may be isolated by means of molecular biological standard techniques and the sequence information provided here. For example, a C. glutamicum RRP cDNA may be isolated from a C. glutamicum bank by using a complete sequence from Appendix A or a section thereof as hybridization probe and by using standard hybridization techniques (as described, for example, in Sambrook, J., Fritsch, E. F. and Maniatis, T. Molecular Cloning: A Laboratory Manual. 2nd Ed., Cold Spring Harbor Laboratory, Cold Spring Harbor Laboratory Press, Cold Spring Harbor, N.Y., 1989). Moreover, a nucleic acid molecule comprising a complete sequence from Appendix A or a section thereof can be isolated via polymerase chain reaction, using the oligonucleotide primers produced on the basis of said sequence (for example, it is possible to isolate a nucleic acid molecule comprising a complete sequence from Appendix A or a section thereof via polymerase chain reaction by using oligonucleotide primers which have been produced on the basis of this same sequence of Appendix A). For example, mRNA can be isolated from normal endothelial cells (for example via the guanidinium thiocyanate extraction method of Chirgwin et al. (1979) Biochemistry 18: 5294-5299), and the cDNA can be prepared by means of reverse transcriptase (e.g. Moloney-MLV reverse transcriptase, available from Gibco/BRL, Bethesda, Md., or AMV reverse transcriptase, available from Seikagaku America, Inc., St. Petersburg, Fla.) and by means of random polynucleotide primers or oligonucleotide primers based on any of the nucleotide sequences shown in Appendix A. Synthetic oligonucleotide primers for amplification via polymerase chain reaction can be produced on the basis of any of the nucleotide sequences shown in Appendix A. A nucleic acid of the invention may be amplified by means of cDNA or, alternatively, genomic DNA as template and of suitable oligonucleotide primers according to PCR standard amplification techniques. The nucleic acid amplified in this way may be cloned into a suitable vector and characterized by DNA sequence analysis. Oligonucleotides corresponding to an RRP nucleotide sequence may further be prepared by standard syntheses using, for example, an automatic DNA synthesizer.

In a preferred embodiment, an isolated nucleic acid molecule of the invention comprises one of the nucleotide sequences listed in Appendix A. The sequences of Appendix A correspond to the Corynebacterium glutamicum RRP cDNAs of the invention. These cDNAs comprise sequences, the RRP proteins (i.e. the “coding region” indicated in each sequence in Appendix A), and also the 5′ and 3′ untranslated sequences likewise indicated in Appendix A. As an alternative, the nucleic acid molecule may comprise only the coding region of any of the sequences in Appendix A.

In addition to this, the nucleic acid molecule of the invention may comprise only a section of the coding region of any of the sequences in Appendix A, for example a fragment which may be used as probe or primer or fragment which encodes a biologically active section of an RRP protein. The nucleotide sequences determined from cloning the RRP genes from C. glutamicum make it possible to generate probes and primers which are designed for identifying and/or cloning RRP homologs in other cell types and organisms and RRP homologs of other corynebacteria or related species. The probe or primer usually comprises essentially purified oligonucleotide. The oligonucleotide usually comprises a nucleotide sequence region which hybridizes under stringent conditions to at least about 12, preferably about 25, more preferably about 40, 50 or 75 successive nucleotides of a sense strand of any of the sequences indicated in Appendix A, of an antisense strand of any of the sequences indicated in Appendix A or naturally occurring mutants thereof. Primers based on a nucleotide sequence of Appendix A may be used in PCR reactions for cloning RRP homologs. Probes based on the RRP nucleotide sequences may be used for detecting transcripts or genomic sequences encoding the same protein or homologous proteins. In preferred embodiments, the probe moreover comprises a labeling group bound thereto, for example a radioisotope, a fluorescent compound, an enzyme or an enzyme cofactor. These probes may be used as part of a diagnostic assay kit for identifying cells which misexpress an RRP protein, for example by measuring an amount of an RRP-encoding nucleic acid in a cell sample, for example detecting the RRP mRNA levels, or by determining whether a genomic RRP gene has been mutated or deleted.

In one embodiment, the nucleic acid molecule of the invention encodes a protein or a section thereof comprising an amino acid sequence which is sufficiently homologous to an amino acid sequence of Appendix B for the protein or a section thereof to retain the ability to modulate the yield, production and/or efficiency of production of one or more fine chemicals in C. glutamicum, to degrade hydrocarbons, to oxidize terpenoids, to serve as a target for drug development and to serve as identification marker for C. glutamicum or related organisms. The term “sufficiently homologous”, as used herein, relates to proteins or sections thereof whose amino acid sequences have a minimum number of identical or equivalent (for example an amino acid residue having a side chain similar to that of an amino acid residue in any of the sequences of Appendix B) amino acid residues compared to an amino acid sequence of Appendix B so that the protein or a section thereof can modulate the yield, production and/or efficiency of production of one or more fine chemicals in C. glutamicum, degrade hydrocarbons, oxidize terpenoids, serve as a target for drug development or serve as identification marker for C. glutamicum or related organisms. Examples of these activities are likewise described herein. Thus, the “function of an RRP protein” contributes to the overall regulation of the metabolic pathway of one or more fine chemicals or to the degradation of a hydrocarbon or to the oxidation of a terpenoid.

Sections of proteins encoded by the RRP nucleic acid molecules of the invention are preferably biologically active sections of any of the RRP proteins. The term “biologically active section of an RRP protein”, as used herein, is intended to comprise a section, for example a domain or a motif, of an RRP protein, which modulates the yield, production and/or efficiency of production of one or more fine chemicals in C. glutamicum, degrades hydrocarbons, oxidizes terpenoids, and serves as a target for drug development or as identification marker for C. glutamicum or related organisms. In order to determine whether an RRP protein or a biologically active section thereof is able to modulate the yield, production and/or efficiency of production of one or more fine chemicals in C. glutamicum, to degrade hydrocarbons or to oxidize terpenoids, an enzyme activity assay may be carried out. These assay methods, as described in detail in example 8 of the examples, are familiar to the skilled worker.

Additional nucleic acid fragments encoding biologically active sections of an RRP protein can be prepared by isolating a section of any of the sequences in Appendix B, expressing the encoded section of the RRP protein or RRP peptide (e.g. by recombinant expression in vitro) and determining the activity of the encoded section of said RRP protein or peptide.

Moreover, the invention comprises nucleic acid molecules which differ from any of the nucleotide sequences shown in Appendix A (and sections thereof) due to the degeneracy of the genetic code and thus encode the same RRP protein as the one encoded by the nucleotide sequences shown in Appendix A. In another embodiment, an isolated nucleic acid molecule of the invention has a nucleotide sequence which encodes a protein having an amino acid sequence shown in Appendix B. In a further embodiment, the nucleic acid molecule of the invention encodes a full-length C. glutamicum protein which is essentially homologous to an amino acid sequence of Appendix B (encoded by an open reading frame shown in Appendix A).

In addition to naturally occurring variants of the RRP sequence, which may exist in the population, the skilled worker is likewise aware of the fact that it is possible to introduce changes into a nucleotide sequence of Appendix A by mutation, leading to a change in the amino acid sequence of the encoded RRP protein without impairing the functionality of said RRP protein. It is possible, for example, to produce nucleotide substitutions in a sequence of Appendix A, which lead to amino acid substitutions at “nonessential” amino acid residues. A “nonessential” amino acid residue is a residue which can be modified in the wild-type sequence of any of the RRP-proteins (Appendix B) without modification of the activity of said RRP protein, whereas an “essential” amino acid residue is required for RRP-protein activity. However, other amino acid residues (e.g. nonconserved or merely semiconserved amino acid residues in the domain with RRP activity) may not be essential for activity and thus can probably be modified without modification of the RRP activity.

Consequently, a further aspect of the invention relates to nucleic acid molecules which encode RRP proteins containing the modified amino acid residues which are nonessential for RRP activity. The amino acid sequence of these RRP proteins differs from a sequence in Appendix B, but said proteins nevertheless retain at least one of the RRP activities described herein. In one embodiment, the isolated nucleic acid molecule comprises a nucleotide sequence which encodes a protein comprising an amino acid sequence which is at least about 50% homologous to an amino acid sequence of Appendix B, which protein can modulate the yield, production and/or efficiency of production of one or more fine chemicals in C. glutamicum, degrade hydrocarbons, oxidize terpenoids, serve as a target for drug development or serve as identification marker for C. glutamicum or related organisms.

An isolated nucleic acid molecule encoding an RRP protein which is homologous to a protein sequence of Appendix B may be generated by introducing one or more nucleotide substitutions, additions or deletions into a nucleotide sequence of Appendix A so that one or more amino acid substitutions, additions or deletions are introduced into the encoded protein. The mutations may be introduced into any of the sequences of Appendix A by standard techniques such as site-directed mutagenesis and PCR-mediated mutagenesis. Preference is given to introducing conservative amino acid substitutions at one or more of the predicted nonessential amino acid residues. A “conservative amino acid substitution” replaces the amino acid residue by an amino acid residue with a similar side chain. Families of amino acid residues with similar side chains have been defined in the art. These families comprise amino acids with basic side chains (e.g. lysine, arginine, histidine), acidic side chains (e.g. aspartic acid, glutamic acid), uncharged polar side chains (e.g. glycine, asparagine, glutamine, serine, threonine, tyrosine, cysteine), nonpolar side chains (e.g. alanine, valine, leucine, isoleucine, proline, phenylalanine, methionine, tryptophan), beta-branched side chains (e.g. threonine, valine, isoleucine) and aromatic side chains (e.g. tyrosine, phenylalanine, tryptophan, histidine). A predicted nonessential amino acid residue in an RRP protein is thus preferably replaced by another amino acid residue of the same side-chain family. In a further embodiment, the mutations may alternatively be introduced randomly over the entire or over part of the RRP-encoding sequence, for example by saturation mutagenesis, and the resulting mutants may be tested for an RRP activity described herein in order to identify mutants maintaining RRP activity. After mutagenesis of any of the sequences of Appendix A, the encoded protein may be expressed recombinantly, and the activity of said protein may be determined, for example, using the assays described herein (see example 8 of the examples).

B. Recombinant Expression Vectors and Host Cells

A further aspect of the invention relates to vectors, preferably expression vectors, containing a nucleic acid which encodes an RRP protein (or a section thereof). The term “vector” as used herein, relates to a nucleic acid molecule capable of transporting another nucleic acid to which it is bound. One type of vector is a “plasmid” which term means a circular double-stranded DNA loop into which additional DNA segments can be ligated. Another type of vector is a viral vector, and here additional DNA segments can be ligated into the viral genome. Certain vectors are capable of replicating autonomously in a host cell into which they have been introduced (for example bacterial vectors with bacterial origin of replication and episomal mammalian vectors). Other vectors (e.g. nonepisomal mammalian vectors) are integrated into the genome of a host cell when introduced into said host cell and thereby replicated together with the host genome. Moreover, particular vectors are capable of controlling the expression of genes to which they are functionally linked. These vectors are referred to herein as “expression vectors”. Normally, expression vectors which can be used in DNA recombination techniques are in the form of plasmids. In the present description, “plasmid” and “vector” may be used interchangeably, since the plasmid is the most commonly used type of vector. The present invention is intended, however, to comprise other types of expression vectors such as viral vectors (for example replication-deficient retroviruses, adenoviruses and adenovirus-related viruses), which exert similar functions.

The recombinant expression vectors of the invention comprise a nucleic acid of the invention in a form which is suitable for expressing said nucleic acid in a host cell, i.e. that the recombinant expression vectors comprise one or more regulatory sequences which are selected on the basis of the host cells to be used for expression and which are functionally linked to the nucleic acid sequence to be expressed. In a recombinant expression vector, the term “functionally linked” means that the nucleotide sequence of interest is bound to the regulatory sequence(s) such that expression of said nucleotide sequence is possible (for example in an in vitro transcription/translation system or in a host cell, if the vector has been introduced into said host cell). The term “regulatory sequence” is intended to comprise promoters, repressor-binding sites, activator-binding sites, enhancer regions and other expression control elements (e.g. terminators, other elements of the mRNA secondary structure or polyadenylation signals). These regulatory sequences are described, for example, in Goeddel: Gene Expression Technology: Methods in Enzymology 185, Academic Press, San Diego, Calif. (1990). Regulatory sequences comprise those which control expression of a nucleotide sequence in many types of host cells and those which control direct expression of the nucleotide sequence only in particular host cells. The skilled worker understands that designing an expression vector may depend on factors such as the choice of host cell to be transformed, the extent of protein expression desired, etc. The expression vectors of the invention may be introduced into the host cells so as to prepare proteins or peptides, including the fusion proteins or fusion peptides, which are encoded by the nucleic acids as described herein (e.g. RRP proteins, mutated forms of RRP proteins, fusion proteins, etc.).

The recombinant expression vectors of the invention may be designed for expressing RRP proteins in prokaryotic or eukaryotic cells. For example, RRP genes may be expressed in bacterial cells such as C. glutamicum, insect cells (using baculovirus expression vectors), yeast cells and other fungal cells (see Romanos, M. A. et al. (1992) “Foreign gene expression in yeast: a review”, Yeast 8: 423-488; van den Hondel, C. A. M. J. J. et al. (1991) “Heterologous gene expression in filamentous fungi” in: More Gene Manipulations in Fungi, J. W. Bennet & L. L. Lasure, Editors, pp. 396-428: Academic Press: San Diego; and van den Hondel, C. A. M. J. J. & Punt, P. J. (1991) “Gene transfer systems and vector development for filamentous fungi in: Applied Molecular Genetics of Fungi, Peberdy, J. F. et al., Editors, pp. 1-28, Cambridge University Press: Cambridge), algal cells and cells of multicellular plants (see Schmidt, R. and Willmitzer, L. (1988) “High efficiency Agrobacterium tumefaciens-mediated transformation of Arabidopsis thaliana leaf and cotyledon explants” Plant Cell Rep.: 583-586) or mammalian cells. Suitable host cells are further discussed in Goeddel, Gene Expression Technology: Methods in Enzymology 185, Academic Press, San Diego, Calif. (1990). As an alternative, the recombinant expression vector may be transcribed and translated in vitro, for example by using T7 promoter regulatory sequences and T7 polymerase.

Proteins are expressed in prokaryotes mainly by using vectors containing constitutive or inducible promoters which control expression of fusion or nonfusion proteins. Fusion vectors control a number of amino acids to a protein encoded therein, usually at the amino terminus of the recombinant protein. These fusion vectors usually have three tasks: 1) enhancing the expression of recombinant protein; 2) increasing the solubility of the recombinant protein; and 3) supporting the purification of the recombinant protein by acting as a ligand in affinity purification. Often a proteolytic cleavage site is introduced into fusion expression vectors at the junction of fusion unit and recombinant protein so that the recombinant protein can be separated from the fusion unit after purifying the fusion protein. These enzymes and their corresponding recognition sequences comprise factor Xa, thrombin and enterokinase.

Common fusion expression vectors comprise PGEX (Pharmacia Biotech Inc; Smith, D. B. and Johnson, K. S. (1988) Gene 67: 31-40), pMAL (New England Biolabs, Beverly, Mass.) und PRIT 5 (Pharmacia, Piscataway, N.J.), in which glutathione S-transferase (GST), maltose E-binding protein and protein A, respectively, are fused to the recombinant target protein. In one embodiment, the coding sequence of the RRP protein is cloned into a PGEX expression vector such that a vector is generated, which encodes a fusion protein comprising, from N terminus to C terminus, GST—thrombin cleavage site—protein X. The fusion protein may be purified via affinity chromatography by means of a glutathione-agarose resin. The recombinant RRP protein which is not fused to GST may be obtained by cleaving the fusion protein with thrombin.

Examples of suitable inducible nonfusion E. coli expression vectors include pTrc (Amann et al., (1988) Gene 69: 301-315) and pET 11d (Studier et al. Gene Expression Technology: Methods in Enzymology 185, Academic Press, San Diego, Calif. (1990) 60-89). The target gene expression from the pTrc vector is based on transcription from a hybrid trp-lac fusion promoter by host RNA polymerase. The target gene expression from the pET11d vector is based on transcription from a T7-gn10-lac fusion promoter, which is mediated by a coexpressed viral RNA polymerase (T7 gn1). This viral polymerase is provided in the BL 21 (DE3) or HMS174 (DE3) host strain by a resident λ prophage which harbors a T7 gn1 gene under the transcriptional control of the lacUV 5 promoter.

One strategy for maximizing expression of the recombinant protein is to express said protein in a host bacterium whose ability to proteolytically cleave said recombinant protein is disrupted (Gottesman, S. Gene Expression Technology: Methods in Enzymology 185, Academic Press, San Diego, Calif. (1990) 119-128). Another strategy is to modify the nucleic acid sequence of the nucleic acid to be inserted into an expression vector such that the individual codons for each amino acid are those which are preferably used in a bacterium selected for expression, such as C. glutamicum (Wada et al. (1992) Nucleic Acids Res. 20: 2111-2118). This modification of the nucleic acid sequences of the invention can be carried out by standard techniques of DNA synthesis.

In a further embodiment, the RRP-protein expression vector is a yeast expression vector. Examples of vectors for expression in the yeast S. cerevisiae include pYepSec1 (Baldari et al., (1987) Embo J. 6: 229-234), pMFa (Kurjan and Herskowitz (1982) Cell 30: 933-943), pJRY88 (Schultz et al. (1987) Gene 54: 113-123) and pYES2 (Invitrogen Corporation, San Diego, Calif.). Vectors and methods for constructing vectors which are suitable for use in other fungi such as filamentous fungi include those which are described in detail in: van den Hondel, C. A. M. J. J. & Punt, P. J. (1991) “Gene transfer systems and vector development for filamentous fungi, in: Applied Molecular Genetics of Fungi, J. F. Peberdy et al., Editors, pp. 1-28, Cambridge University Press: Cambridge.

As another alternative, it is possible to express the RRP proteins of the invention in insect cells using baculovirus expression vectors. Baculovirus vectors available for expression of proteins in cultured insect cells (e.g. Sf9 cells) include the pAc series (Smith et al., (1983) Mol. Cell Biol. 3: 2156-2165) and the pVL series (Lucklow and Summers (1989) Virology 170: 31-39).

In a further embodiment, the RRP proteins of the invention may be expressed in cells of unicellular plants (such as algae) or in cells of the higher plants (e.g. spermatophytes such as crops). Examples of expression vectors of plants include those which are described in detail in: Bekker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acids Res. 12: 8711-8721.

A further embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6: 187-195). When used in mammalian cells, the control functions of the expression vector are often provided by viral regulatory elements. Commonly used promoters are derived, for example, from polyoma, adenovirus 2, cytomegalovirus and simian virus 40. Other suitable expression systems for prokaryotic and eukaryotic cells can be found in chapters 16 and 17 by Sambrook, J., Fritsch, E. F. and Maniatis, T., Molecular cloning: A Laboratory Manual, 2nd Edition, Cold Spring Harbor Laboratory, Cold Spring Harbor Laboratory Press, Cold Spring Harbor, N.Y., 1989.

In a further embodiment, the recombinant mammalian expression vector may preferably cause expression of the nucleic acid in a particular cell type (for example, tissue-specific regulatory elements are used for expressing the nucleic acid). Tissue-specific regulatory elements are known in the art. Non-limiting examples of suitable tissue-specific promoters include the albumin promoter (liver-specific; Pinkert et al. (1987) Genes Dev. 1: 268-277), lymphoid-specific promoters (Calame und Eaton (1988) Adv. Immunol. 43: 235-275), in particular promoters of T-cell receptors (Winoto and Baltimore (1989) EMBO J. 8: 729-733) and immunoglobulins (Banerji et al. (1983) Cell 33: 729-740; Queen and Baltimore (1983) Cell 33: 741-748), neuron-specific promoters (e.g. the neurofilament promoter; Byrne and Ruddle (1989) PNAS 86: 5473-5477), pancreas-specific promoters (Edlund et al., (1985) Science 230: 912-916) and mamma-specific promoters (e.g. milk serum promoter; U.S. Pat. No. 4,873,316 and European Patent Application document No. 264 166). Development-regulated promoters for example the murine hox promoters (Kessel and Gruss (1990) Science 249: 374-379) and the α-fetoprotein promoter (Campes and Tilghman (1989) Genes Dev. 3: 537-546), are likewise included.

Moreover, the invention provides a recombinant expression vector comprising an inventive DNA molecule which has been cloned into the expression vector in antisense direction. This means that the DNA molecule is functionally linked to a regulator sequence such that expression of an RNA molecule which is antisense to RRP mRNA becomes possible (via transcription of the DNA molecule). It is possible to select regulatory sequences which are functionally bound to a nucleic acid cloned in antisense direction and which control continuous expression of the antisense RNA molecule in a multiplicity of cell types; for example, it is possible to select viral promoters and/or enhancers or regulatory sequences which control the constitutive tissue-specific or cell type-specific expression of antisense RNA. The antisense expression vector may be in the form of a recombinant plasmid, phagemid or attenuated virus and produces antisense nucleic acids under the control of a highly effective regulatory region whose activity is determined by the cell type into which the vector is introduced. The regulation of gene expression by means of antisense genes is discussed in Weintraub, H. et al., Antisense—RNA as a molecular tool for genetic analysis, Reviews—Trends in Genetics, Vol. 1(1) 1986.

A further aspect of the invention relates to the host cells into which a recombinant expression vector of the invention has been introduced. The terms “host cell” and “recombinant host cell” are used interchangeably herein. Naturally, these terms relate not only to a particular target cell but also to the progeny or potential progeny of this cell. Since particular modifications may appear in successive generations, due to mutation or environmental factors, this progeny is not necessarily identical to the parental cell but is still included within the scope of the term as used herein.

A host cell may be a prokaryotic or eukaryotic cell. For example, an RRP protein may be expressed in bacterial cells such as C. glutamicum, insect cells, yeast cells or mammalian cells (such as Chinese hamster ovary (CHO) cells or COS cells). Other suitable host cells are familiar to the skilled worker. Microorganisms which are related to Corynebacterium glutamicum and can be used in a suitable manner as host cells for the nucleic acid and protein molecules of the invention are listed in Table 3.

Conventional transformation or transfection methods can be used to introduce vector DNA into prokaryotic or eukaryotic cells. The terms “transformation” and “transfection”, “conjugation” and “transduction”, as used herein, are intended to comprise a multiplicity of methods known in the art for introducing foreign nucleic acid (e.g. DNA) into a host cell, including natural competence, chemically mediated transfer, calcium phosphate or calcium chloride coprecipitation, DEAE dextran-mediated transfection, lipofection or electroporation. Suitable methods for transformation or transfection of host cells can be found in Sambrook et al. (Molecular Cloning: A Laboratory Manual. 2nd Edition, Cold Spring Harbor Laboratory, Cold Spring Harbor Laboratory Press, Cold Spring Harbor, N.Y., 1989) and other laboratory manuals.

In the case of stable transfection of mammalian cells, it is known that, depending on the expression vector used and the transfection technique used, only a small proportion of the cells can integrate the foreign DNA into their genome. These integrants are usually identified and selected by introducing a gene which encodes a selectable marker (e.g. resistant to antibiotics) together with the gene of interest into the host cells. Preferred selectable markers include those which impart resistance to drugs such as G418, hygromycin and methotrexate. A nucleic acid which encodes a selectable marker may be introduced into a host cell on the same vector that encodes an RRP protein or may be introduced in a separate vector. Cells which have been stably transfected with the introduced nucleic acid may be identified for example by drug selection (for example, cells which have integrated the selectable marker survive, whereas the other cells die).

A homologous recombined microorganism is generated by preparing a vector which contains at least one RRP-gene section into which a deletion, addition or substitution has been introduced in order to modify, for example functionally disrupt, the RRP gene. Said RRP gene is preferably a Corynebacterium glutamicum RRP gene, but it is also possible to use a homolog from a related bacterium or even from a mammalian, yeast or insect source. In a preferred embodiment, the vector is designed such that homologous recombination functionally disrupts the endogenous RRP gene (i.e., the gene no longer encodes a functional protein; also referred to as “knockout” vector). As an alternative, the vector may be designed such that homologous recombination mutates or otherwise modifies the endogenous RRP gene which, however, still encodes the functional protein (for example, the regulatory region located upstream may be modified such that thereby expression of the endogenous RRP protein is modified.). The modified RRP-gene fraction in the homologous recombination vector is flanked at its 5′ and 3′ ends by additional nucleic acids of the RRP gene, which makes possible a homologous recombination between the exogenous RRP gene carried by the vector and an endogenous RRP gene in a microorganism. The length of the additional flanking RRP nucleic acid is sufficient for a successful homologous recombination with the endogenous gene. Usually, the vector contains less than one kilobase of flanking DNA (both at the 5′ and the 3′ ends) (see, for example, Thomas, K. R. and Capecchi, M. R. (1987) Cell 51: 503, for a description of homologous recombination vectors). The vector is introduced into a microorganism (e.g. by electroporation) and cells in which the introduced RRP gene has homologously recombined with the endogenous RRP gene are selected using methods known in the art.

In another embodiment, it is possible to produce recombinant microorganisms which contain selected systems which make possible a regulated expression of the introduced gene. The insertion of an RRP gene in a vector, as a result of which it is brought under the control of the lac operon, enables, for example, RRP-gene expression only in the presence of IPTG. These regulatory systems are known in the art.

A host cell of the invention, such as a prokaryotic or eukaryotic host cell in culture, may be used for producing (i.e. expressing) an RRP protein. Moreover, the invention provides methods for producing RRP proteins by using the host cells of the invention. In one embodiment, the method comprises the cultivation of the host cell of the invention (into which a recombinant expression vector encoding an RRP protein has been introduced or in whose genome a gene encoding a wild-type or modified RRP protein has been introduced) in a suitable medium until the RRP protein has been produced. In a further embodiment, the method comprises isolating the RRP proteins from the medium or the host cell.

C. Uses and Methods of the Invention

The nucleic acid molecules, proteins, protein homologs, fusion proteins, primers, vectors and host cells described herein may be used in one or more of the following methods: identification of C. glutamicum and related organisms, mapping of genomes of organisms related to C. glutamicum, identification and localization of C. glutamicum sequences of interest, evolutionary studies, determination of RRP-protein regions required for function, modulation of the activity of an RRP protein; modulation of the activity of one or more metabolic pathways and modulation of the cellular production of a compound of interest, such as a fine chemical. The RRP nucleic acid molecules of the invention have a multiplicity of uses. First, they may be used for identifying an organism as Corynebacterium glutamicum or close relatives thereof. They may also be used for identifying the presence of C. glutamicum or a relative thereof in a mixed population of microorganisms. The invention provides the nucleic acid sequences of a number of C. glutamicum genes. Probing the extracted genomic DNA of a culture of a uniform or mixed population of microorganisms under stringent conditions with a probe which covers a region of a C. glutamicum gene which is unique for this organism makes it possible to determine whether said organism is present. Although Corynebacterium glutamicum itself is nonpathogenic, it is related to pathogenic species such as Corynebacterium diphtheriae. The detection of such an organism is of substantial clinical importance.

The presence of C. glutamicum in a sample can be detected by using techniques known in the art. In particular, the cells in the sample may first be grown in a suitable liquid or on a suitable solid culture medium in order to increase the number of cells in the culture. Said cells are lysed and all of the DNA contained therein is extracted and, where appropriate, purified in order to remove cell debris and protein material, which could interfere with the subsequent analysis. A polymerase chain reaction or a similar technique known in the art is carried out (a general overview over methodologies usually used for nucleic acid sequence amplification can be found in Mullis et al., U.S. Pat. No. 4,683,195, Mullis et al., U.S. Pat. No. 4,965,188 and Innis, M. A. and Gelfand, D. H. (1989) PCR-Protocols, A guide to Methods and Applications, Academic Press, pp. 3-12, and (1988) Biotechnology 6:1197 and international Patent Application No. WO89/01050), with primers which are specific for an RRP nucleic acid molecule of the invention being incubated with the nucleic acid sample so that said particular RRP nucleic acid sequence is amplified, if present in the sample. The particular nucleic acid sequence to be amplified is selected on the basis of its exclusive presence in the genome of C. glutamicum and of only a few closely related bacteria. The presence of the desired amplification product indicates the presence of C. glutamicum or an organism closely related to C. glutamicum.

The nucleic acid and protein molecules of the invention may further serve as markers for specific regions of the genome. Using techniques known in the art, it is possible to detect the physical location of the RRP nucleic acid molecules of the invention on the C. glutamicum genome, and this may be used in turn to localize more easily other nucleic acid molecules and genes on the map. Moreover, the nucleic acid molecules of the invention may be sufficiently homologous to the sequences of related species for these nucleic acid molecules to likewise enable the construction of a genomic map in such bacteria (e.g. Brevibacterium lactofermentum).

The nucleic acid and protein molecules of the invention are suitable not only for mapping the genome but also for functional studies of C. glutamicum proteins. The genome region to which a particular C. glutamicum DNA-binding protein binds may be identified by cleaving, for example, the C. glutamicum genome and incubating the fragments with said DNA-binding protein. Those fragments which bind to the protein may additionally be probed with the nucleic acid molecules of the invention, preferably by using readily detectable labels; binding of such a nucleic acid molecule to the genomic fragment enables the localization of said fragment on the genomic map of C. glutamicum, and this facilitates a rapid determination of the nucleic acid sequence to which the protein binds, if carried out several times using different enzymes.

The RRP nucleic acid molecules of the invention are likewise suitable for evolutionary studies and protein structure studies. A multiplicity of prokaryotic and eukaryotic cells utilize the metabolic processes in which the molecules of the invention are involved; it is possible, by comparing the sequences of the nucleic acid molecules of the invention with those encoding similar enzymes from other organisms, to determine the degree of evolutionary relatedness of said organisms. Accordingly, such a comparison makes it possible to determine which sequences are conserved and which are not, and this may be helpful in determining those protein regions which are essential for enzyme function. This type of determination is valuable for protein engineering studies and may indicate how much mutagenesis said protein can tolerate without losing its function.

The RRP proteins of the invention can be used as markers for classifying an unknown bacterium as C. glutamicum or for identifying C. glutamicum or closely related bacteria in a sample. Using techniques known in the art, it is possible, for example, to amplify, where appropriate, cells in a sample (for example by cultivation in a suitable medium) in order to increase the size of the sample, and then to lyse said cells so that the proteins contained therein are released. Said sample may be purified, where appropriate, in order to remove cell debris and nucleic acid molecules, which could interfere with the subsequent analysis. Antibodies specific for a selected RRP protein of the invention may be incubated with the protein sample in a typical Western assay format (see, for example, Ausubel et al., (1988) Current Protocols in Molecular Biology, Wiley: N.Y.), with the antibody binding to its target protein, if this protein is present in the sample. An RRP protein is selected for this type of assay if it is unique or almost unique in C. glutamicum or in C. glutamicum and very closely related bacteria. The proteins in the sample are then fractionated by gel electrophoresis and transferred to a suitable matrix such as nitrocellulose. A suitable secondary antibody with a detectable label (e.g. chemiluminescent or calorimetric) is incubated with said matrix, followed by stringent washing. The presence or absence of the label indicates the presence or absence of the target protein in the sample. If the protein is present, this indicates the presence of C. glutamicum. A similar method makes it possible to classify an unknown bacterium as C. glutamicum; this bacterium is probably not C. glutamicum if a number of C. glutamicum-specific proteins are not detected in the protein samples which have been prepared from said unknown bacterium.

Genetic manipulation of the RRP nucleic acid molecules of the invention can produce RRP proteins with functional differences compared to the wild-type RRP proteins. These proteins may be improved with regard to their efficiency or activity, may be present in the cell in a larger amount than usual or may be weakened with regard to their efficiency or activity.

These changes in activity may directly moderate the yield, production and/or efficiency of production of one or more fine chemicals in C. glutamicum. It is possible, for example, by modifying the activity of a protein involved in the biosynthesis or degradation of a fine chemical (i.e. by mutating the corresponding gene) to directly modulate the ability of the cell to synthesize or degrade this compound and thereby to modulate the yield and/or efficiency of production of said fine chemical. Likewise, it is possible, by modulating the activity of a protein which regulates a metabolic pathway of a fine chemical, to have a direct influence on whether production of the compound of interest is up- or downregulated, with both of these processes modulating the yield or efficiency of production of the fine chemical from the cell.

Modification of the activity of a protein of the invention (i.e. mutagenesis of the corresponding gene) may also modulate indirectly the production of fine chemicals so that the ability of the cell to grow and to divide or to remain viable and productive is increased overall. Fine chemicals are usually produced from C. glutamicum by a large-scale fermentative culture of these microorganisms under conditions which frequently are suboptimal for growth and cell division. It may be possible, by modifying a protein of the invention (e.g. a stress reaction protein, a cell wall protein or a protein involved in the metabolism of compounds required for cell growth and cell division to take place, such as nucleotides and amino acids) so that better survival, growth and propagation is possible under said conditions, to increase the number and productivity of these modified C. glutamicum cells in large-scale culture, and this should in turn lead to increased yields and/or increased efficiency of production of one or more fine chemicals of interest. Furthermore, the metabolic pathways of a cell are necessarily dependent on one another and coregulated. It is possible, by changing the activity of any metabolic pathway in C. glutamicum (i.e. by changing the activity of any of the proteins of the invention, which are involved in such a pathway), to change simultaneously the activity or regulation of another metabolic pathway in said microorganism, which may be involved directly in the synthesis or degradation of a fine chemical.

These abovementioned strategies for the mutagenesis of RRP proteins, which ought to increase the yields of a fine chemical in C. glutamicum, are not intended to be limiting; variations of these mutagenesis strategies are quite obvious to the skilled worker. By using these strategies and including the mechanisms disclosed herein, it is possible to use the nucleic acid and protein molecules of the invention in order to generate C. glutamicum or related bacterial strains expressing mutated RRP nucleic acids and protein molecules so as to improve the yield, production and/or efficiency of production of a compound of interest. The compound of interest may be any product produced by C. glutamicum including the end products of biosynthetic pathways and intermediates of naturally occurring metabolic pathways and also molecules which do not naturally occur in the C. glutamicum metabolism but are produced by a C. glutamicum strain of the invention.

The following examples which are not to be understood as being limiting further illustrate the present invention. The contents of all references, patent applications, patents and published patent applications cited in this patent application are hereby incorporated by way of reference.

### EXAMPLES

### Example 1

### Preparation of Total Genomic DNA from Corynebacterium glutamicum ATCC13032

A Corynebacterium glutamicum (ATCC 13032) culture was cultivated with vigorous shaking in BHI medium (Difco) at 30° C. overnight. The cells were harvested by centrifugation, the supernatant was discarded and the cells were resuspended in 5 ml of buffer I (5% of the original culture volume—all volumes stated have been calculated for a culture volume of 100 ml). Composition of buffer I: 140.34 g/l sucrose, 2.46 g/l MgSO₄·7 H₂O, 10 ml/l KH₂PO₄ solution (100 g/l, adjusted to pH 6.7 with KOH), 50 ml/l M12 concentrate (10 g/l (NH₄)₂SO₄, 1 g/l NaCl, 2 g/l MgSO₄·7 H₂O, 0.2 g/l CaCl₂, 0.5 g/l yeast extract (Difco), 10 ml/l trace element mixture (200 mg/l FeSO₄·H₂O, 10 mg/l ZnSO₄·7 H₂O, 3 mg/l MnCl₂·4 H₂O, 30 mg/l H₃BO₃, 20 mg/l CoCl₂·6 H₂O, 1 mg/l NiCl₂·6 H₂O, 3 mg/l Na₂MoO₄·2 H₂O), 500 mg/l complexing agents (EDTA or citric acid), 100 ml/l vitamin mixture (0.2 ml/l biotin, 0.2 mg/l folic acid, 20 mg/l p-aminobenzoic acid, 20 mg/l riboflavin, 40 mg/l Ca panthothenate, 140 mg/l nicotinic acid, 40 mg/l pyridoxal hydrochloride, 200 mg/l myoinositol). Lysozyme was added to the suspension at a final concentration of 2.5 mg/ml. After incubation at 37° C. for approx. 4 h, the cell wall was degraded and the protoplasts obtained were harvested by centrifugation. The pellet was washed once with 5 ml of buffer I and once with 5 ml of TE buffer (10 mM Tris-HCl, 1 mM EDTA, pH 8). The pellet was resuspended in 4 ml of TE buffer and 0.5 ml of SDS solution (10%) and 0.5 ml of NaCl solution (5 M) were added. After addition of proteinase K at a final concentration of 200 μg/ml, the suspension was incubated at 37° C. for approx. 18 h. The DNA was purified via extraction with phenol, phenol/chloroform/isoamyl alcohol and chloroform/isoamyl alcohol by means of standard methods. The DNA was then precipitated by addition of 1/50 volume of 3 M sodium acetate and 2 volumes of ethanol, subsequent incubation at −20° C. for 30 min and centrifugation at 12 000 rpm in a high-speed centrifuge using an SS34 rotor (Sorvall) for 30 min. The DNA was dissolved in 1 ml of TE buffer containing 20 μ/g/ml RNase A and dialyzed against 1000 ml of TE buffer at 4° C. for at least 3 h. The buffer was exchanged 3 times during this period. 0.4 ml of 2 M LiCl and 0.8 ml of ethanol were added to 0.4 ml aliquots of the dialyzed DNA solution. After incubation at −20° C. for 30 min, the DNA was collected by centrifugation (13 000 rpm, Biofuge Fresco, Heraeus, Hanau, Germany). The DNA pellet was dissolved in TE buffer. It was possible to use DNA prepared by this method for all purposes, including Southern blotting and constructing genomic libraries.

### Example 2

### Construction of Genomic Corynebacterium glutamicum (ATCC13032) Banks in Escherichia coli

Starting from DNA prepared as described in Example 1, cosmid and plasmid banks were prepared according to known and well-established methods (see, for example, Sambrook, J. et al. (1989) “Molecular Cloning: A Laboratory Manual”. Cold Spring Harbor Laboratory Press or Ausubel, F. M. et al. (1994) “Current Protocols in Molecular Biology”, John Wiley & Sons).

It was possible to use any plasmid or cosmid. Particular preference was given to using the plasmids pBR322 (Sutcliffe, J. G. (1979) Proc. Natl Acad. Sci. USA, 75: 3737-3741); pACYC177 (Change & Cohen (1978) J. Bacteriol. 134: 1141-1156); pBS series plasmids (pBSSK+, PBSSK− and others; Stratagene, LaJolla, USA) or cosmids such as SuperCos1 (Stratagene, LaJolla, USA) or Lorist6 (Gibson, T. J. Rosenthal, A., and Waterson, R. H. (1987) Gene 53: 283-286.

### Example 3

### DNA Sequencing and Functional Computer Analysis

Genomic banks, as described in Example 2, were used for DNA sequencing according to standard methods, in particular the chain termination method using ABI377 sequencers (see, for example, Fleischman, R. D. et al. (1995) “Whole-genome Random Sequencing and Assembly of Haemophilus Influenzae Rd., Science 269; 496-512). Sequencing primers having the following nucleotide sequences were used: 5′-GGAAACAGTATGACCATG-3′ oder 5′-GTAAAACGACGGCCAGT-3′.

### Example 4

### In Vivo Mutagenesis

In vivo mutagenesis of Corynebacterium glutamicum may be carried out by passing a plasmid (or other vector) DNA through E. coli or other microorganisms (e.g. Bacillus spp. or yeasts such as Saccharomyces cerevisiae) which cannot maintain the integrity of their genetic information. Common mutator strains contain mutations in the genes for the DNA repair system (e.g., mutHLS, mutD, mutT, etc., for comparison see Rupp, W. D. (1996) DNA repair mechanisms in Escherichia coli and Salmonella, pp. 2277-2294, ASM: Washington). These strains are known to the skilled worker. The use of these strains is illustrated, for example, in Greener, A. and Callahan, M. (1994) Strategies 7:32-34.

### Example 5

### DNA Transfer Between Escherichia coli and Corynebacterium glutamicum

A plurality of Corynebacterium and Brevibacterium species contain endogenous plasmids (such as, for example, pHM1519 or pBL1) which replicate autonomously (for a review see, for example, Martin, J. F. et al. (1987) Biotechnology 5: 137-146). Shuttle vectors for Escherichia coli and Corynebacterium glutamicum can be constructed readily by means of standard vectors for E. coli (Sambrook, J. et al., (1989), “Molecular Cloning: A Laboratory Manual”, Cold Spring Harbor Laboratory Press or Ausubel, F. M. et al. (1994) “Current Protocols in Molecular Biology”, John Wiley & Sons), to which an origin of replication for and a suitable marker from Corynebacterium glutamicum is added. Such origins of replication are preferably taken from endogenous plasmids which have been isolated from Corynebacterium and Brevibacterium species. Particularly useful transformation markers for these species are genes for kanamycin resistance (such as those derived from the Tn5 or the Tn903 transposon) or for chloramphenicol resistance (Winnacker, E. L. (1987) “From Genes to Clones—Introduction to Gene Technology, VCH, Weinheim). There are numerous examples in the literature for preparing a large multiplicity of shuttle vectors which replicate in E. coli and C. glutamicum and can be used for various purposes, including the overexpression of genes (see, for example, Yoshihama, M. et al. (1985) J. Bacteriol. 162: 591-597, Martin, J. F. et al., (1987) Biotechnology, 5: 137-146 and Eikmanns, B. J. et al. (1992) Gene 102: 93-98).

Standard methods make it possible to clone a gene of interest into one of the above-described shuttle vectors and to introduce such hybrid vectors into Corynebacterium glutamicum strains. C. glutamicum can be transformed via protoplast transformation (Kastsumata, R. et al., (1984) J. Bacteriol. 159, 306-311), electroporation (Liebl, E. et al., (1989) FEMS Microbiol. Letters, 53: 399-303) and, in cases in which specific vectors are used, also via conjugation (as described, for example, in Schäfer, A., et al. (1990) J. Bacteriol. 172: 1663-1666).

Likewise, it is possible to transfer the shuttle vectors for C. glutamicum to E. coli by preparing plasmid DNA from C. glutamicum (by means of standard methods known in the art) and transforming it into E. coli. This transformation step can be carried out using standard methods but advantageously an Mcr-deficient E. coli strain such as NM522 (Gough & Murray (1983) J. Mol. Biol. 166: 1-19) is used.

### Example 6

### Determination of the Expression of the Mutant Protein

The observations of the activity of a mutated protein in a transformed host cell are based on the fact that the mutant protein is expressed in a similar manner and in similar quantity to the wild-type protein. A suitable method for determining the amount of transcription of the mutant gene (an indication of the amount of mRNA available for translation of the gene product) is to carry out a Northern blot (see, for example, Ausubel et al., (1988) Current Protocols in Molecular Biology, Wiley: N.Y.), with a primer which is designed such that it binds to the gene of interest being provided with a detectable (usually radioactive or chemiluminescent) label such that—when the total RNA of a culture of the organism is extracted, fractionated on a gel, transferred to a stable matrix and incubated with this probe-binding and binding quantity of the probe indicate the presence and also the amount of mRNA for said gene. This information is an indication of the extent to which the mutant gene has been transcribed. Total cell RNA can be isolated from Corynebacterium glutamicum by various methods known in the art, as described in Bormann, E. R. et al., (1992) Mol. Microbiol. 6: 317-326.

The presence or the relative amount of protein translated from said mRNA can be determined by using standard techniques such as Western blot (see, for example, Ausubel et al. (1988) “Current Protocols in Molecular Biology”, Wiley, N.Y.). In this method, total cell proteins are extracted, fractionated by gel electrophoresis, transferred to a matrix such as nitrocellulose and incubated with a probe, for example an antibody, which binds specifically to the protein of interest. This probe is usually provided with a chemiluminescent or colorimetric label which can be readily detected. The presence and the observed amount of label indicate the presence and the amount of the desired mutant protein in the cell.

### Example 7

### Growth of Genetically Modified Corynebacterium glutamicum—Media and Cultivation Conditions

Genetically modified corynebacteria are cultivated in synthetic or natural growth media. A number of different growth media for corynebacteria are known and readily available (Lieb et al. (1989) Appl. Microbiol. Biotechnol. 32: 205-210; von der Osten et al. (1998) Biotechnology Letters 11: 11-16; Patent DE 4 120 867; Liebl (1992) “The Genus Corynebacterium”, in: The Procaryotes, Vol. II, Balows, A., et al., editors Springer-Verlag). These media are composed of one or more carbon sources, nitrogen sources, inorganic salts, vitamins and trace elements. Preferred carbon sources are sugars such as mono-, di- or polysaccharides. Examples of very good carbon sources are glucose, fructose, mannose, galactose, ribose, sorbose, ribulose, lactose, maltose, sucrose, raffinose, starch and cellulose. Sugars may also be added to the media via complex compounds such as molasses or other byproducts of sugar refining. It may also be advantageous to add mixtures of various carbon sources. Other possible carbon sources are alcohols and organic acids, such as methanol, ethanol, acetic acid or lactic acid. Nitrogen sources are usually organic or inorganic nitrogen compounds or materials containing these compounds. Examples of nitrogen sources include ammonia gas and ammonium salts such as NH₄Cl or (NH₄)₂SO₄, NH₄OH, nitrates, urea, amino acids and complex nitrogen sources such as cornsteep liquor, soya meal, soya protein, yeast extract, meat extract and others.

Inorganic salt compounds which may be present in the media include the chloride, phosphorus or sulfate salts of calcium, magnesium, sodium, cobalt, molybdenum, potassium, manganese, zinc, copper and iron. Chelating agents may be added to the medium in order to keep the metal ions in solution. Particularly suitable chelating agents include dihydroxyphenols such as catechol or protocatechuate and organic acids such as citric acid. The media usually also contain other growth factors such as vitamins or growth promoters, examples of which include biotin, riboflavin, thiamine, folic acid, nicotinic acid, panthothenate and pyridoxine. Growth factors and salts are frequently derived from complex media components such as yeast extract, molasses, cornsteep liquor and the like. The exact composition of the media heavily depends on the particular experiment and is decided upon individually for each specific case. Information on the optimization of media can be found in the textbook “Applied Microbiol. Physiology, A Practical Approach” (editors P. M. Rhodes, P. F. Stanbury, IRL Press (1997) pp. 53-73, ISBN 0 19 963577 3). Growth media can also be obtained from commercial suppliers, for example Standard 1 (Merck) or BHI (brain heart infusion, DIFCO) and the like.

All media components are sterilized, either by heat (20 min at 1.5 bar and 121° C.) or by sterile filtration. The components may be sterilized either together or, if required, separately. All media components may be present at the start of the cultivation or added continuously or batchwise, as desired.

The cultivation conditions are defined separately for each experiment. The temperature should be between 15° C. and 45° C. and may be kept constant or may be altered during the experiment. The pH of the medium should be in the range from 5 to 8.5, preferably around 7.0 and may be maintained by adding buffers to the media. An example of a buffer for this purpose is a potassium phosphate buffer. Synthetic buffers such as MOPS, HEPES; ACES, etc. may be used alternatively or simultaneously. Addition of NaOH or NH₄OH can also keep the pH constant during cultivation. If complex media components such as yeast extract are used, the demand for additional buffers decreases, since many complex compounds have a high buffer capacity. In the case of using a fermenter for cultivating microorganisms, the pH may also be regulated using gaseous ammonia.

The incubation period is usually in a range from several hours to several days. This time is selected such that the maximum amount of product accumulates in the broth. The growth experiments disclosed may be carried out in a multiplicity of containers such as microtiter plates, glass tubes, glass flasks or glass or metal fermenters of different sizes. For the screening of a large number of clones, the microorganisms should be grown in microtiter plates, glass tubes or shaker flasks either with or without baffles. Preference is given to using 100 ml shaker flasks which are filled with 10% (based on volume) of the required growth medium. The flasks should be shaken on an orbital shaker (amplitude 25 mm) at a speed in the range of 100-300 rpm. Losses due to evaporation can be reduced by maintaining a humid atmosphere; alternatively, the losses due to evaporation should be corrected mathematically.

If genetically modified clones are investigated, an unmodified control clone or a control clone containing the basic plasmid without insert should also be assayed. The medium is inoculated to an OD₆₀₀ of 0.5-1.5, with cells being used which have been grown on agar plates such as CM plates (10 g/l glucose, 2.5 g/l NaCl, 2 g/l urea, 10 g/l polypeptone, 5 g/l yeast extract, 5 g/l eat extract, 22 g/l agar pH 6.8 with 2 M NaOH) which have been incubated at 30° C. The media are inoculated either by introducing a saline solution of C. glutamicum cells from CM plates or by adding a liquid preculture of said bacterium.

### Example 8

### In Vitro Analysis of the Function of Mutant Proteins

The determination of the activities and kinetic parameters of enzymes is well known in the art. Experiments for determining the activity of a particular modified enzyme must be adapted to the specific activity of the wild-type enzyme, and this is within the capabilities of the skilled worker. Overviews regarding enzymes in general and also specific details concerning the structure, kinetics, principles, methods, applications and examples of the determination of many enzyme activities can be found, for example, in the following references: Dixon, M., and Webb, E. C: (1979) Enzymes, Longmans, London; Fersht (1985) Enzyme Structure and Mechanism, Freeman, N.Y.; Walsh (1979) Enzymatic Reaction Mechanisms. Freeman, San Francisco; Price, N. C., Stevens, L. (1982) Fundamentals of Enzymology. Oxford Univ. Press: Oxford; Boyer, P. D: editors (1983) The Enzymes, 3rd edition, Academic Press, New York; Bisswanger, H. (1994) Enzymkinetik, 2nd edition VCH, Weinheim (ISBN 3527300325); Bergmeyer, H. U., Bergmeyer, J., Graβl, M. editors (1983-1986) Methods of Enzymatic Analysis, 3rd edition, Vol. I-XII, Verlag Chemie: Weinheim; and Ullmann's Encyclopedia of Industrial Chemistry (1987) Vol. A9, “Enzymes”, VCH, Weinheim, pp. 352-363.

The activity of proteins binding to DNA can be measured by many well-established methods such as DNA bandshift assays (which are also referred to as gel retardation assays). The action of these proteins on the expression of other molecules can be measured using reporter gene assays (as described in Kolmar, H. et al., (1995) EMBO J. 14: 3895-3904 and in references therein). Reporter gene assay systems are well known and established for applications in prokaryotic and eukaryotic cells, with enzymes such as beta-galactosidase, green fluorescent protein and several other enzymes being used.

The activity of membrane transport proteins can be determined according to techniques as are described in Gennis, R. B. (1989) “Pores, Channels and Transporters”, in Biomembranes, Molecular Structure and Function, Springer: Heidelberg, pp. 85-137; 199-234; and 270-322.

### Example 9

### Analysis of the Influence of Mutated Protein on the Production of the Product of Interest

The effect of the genetic modification in C. glutamicum on the production of a compound of interest (such as an amino acid) can be determined by growing the modified microorganisms under suitable conditions (such as the ones described above) and testing the medium and/or the cellular components with regard to increased production of the product of interest (i.e. an amino acid). Such analytical techniques are well known to the skilled worker and include spectroscopy, thin-layer chromatography, various types of coloring methods, enzymic and microbiological methods and analytical chromatography such as high performance liquid chromatography (see, for example, Ullman, Encyclopedia of Industrial Chemistry, Vol. A2, pp. 89-90 and pp. 443-613, VCH: Weinheim (1985); Fallon, A., et al., (1987) “Applications of HPLC in Biochemistry” in: Laboratory Techniques in Biochemistry and Molecular Biology, Vol. 17; Rehm et al. (1993) Biotechnology, Vol. 3, Chapter III: “Product recovery and purification”, pp. 469-714, VCH: Weinheim; Belter, P. A. et al. (1988) Bioseparations: downstream processing for Biotechnology, John Wiley and Sons; Kennedy, J. F. and Cabral, J. M. S. (1992) Recovery processes for biological Materials, John Wiley and Sons; Shaeiwitz, J. A. and Henry, J. D. (1988) Biochemical Separations, in Ullmann's Encyclopedia of Industrial Chemistry, Vol. B3; Chapter 11, pp. 1-27, VCH: Weinheim; and Dechow, F. J. (1989) Separation and purification techniques in biotechnology, Noyes Publications).

In addition to measuring the end product of the fermentation, it is likewise possible to analyze other components of the metabolic pathways, which are used for producing the compound of interest, such as intermediates and byproducts, in order to determine the overall efficiency of production of the compound. The analytical methods include measuring the amounts of nutrients in the medium (for example sugars, hydrocarbons, nitrogen sources, phosphate and other ions), measuring biomass composition and growth, analyzing the production of common metabolites from biosynthetic pathways and measuring gases generated during fermentation. Standard methods for these measurements are described in Applied Microbial Physiology; A Practical Approach, P. M. Rhodes and P. F. Stanbury, editors IRL Press, pp. 103-129; 131-163 and 165-192 (ISBN: 0199635773) and the references therein.

### Example 10

### Purification of the Product of Interest from a C. glutamicum Culture

The product of interest may be obtained from C. glutamicum cells or from the supernatant of the above-described culture by various methods known in the art. If the product of interest is not secreted by the cells, the cells may be harvested from the culture by slow centrifugation, and the cells may be lysed by standard techniques such as mechanial force or sonication. The cell debris is removed by centrifugation and the supernatant fraction which contains the soluble proteins is obtained for further purification of the compound of interest. If the product is secreted by the C. glutamicum cells, the cells are removed from the culture by slow centrifugation and the supernatant fraction is retained for further purification.

The supernatant fraction from both purification methods is subjected to chromatography using a suitable resin, and either the molecule of interest is retained on the chromatography resin while many contaminants in the sample are not, or the contaminants remain on the resin while the sample does not. If necessary, these chromatography steps can be repeated using the same or different chromatography resins. The skilled worker is familiar with the selection of suitable chromatography resins and the most effective application thereof for a particular molecule to be purified. The purified product may be concentrated by filtration or ultrafiltration and stored at a temperature at which product stability is highest.

In the art, many purification methods are known, and the above purification method is not intended to be limiting. These purification techniques are described, for example, in Bailey, J. E. & Ollis, D. F. Biochemical Engineering Fundamentals, McGraw-Hill: N.Y. (1986).

The identity and purity of the isolated compounds can be determined by techniques of the prior art. These techniques comprise high performance liquid chromatography (HPLC), spectroscopic methods, coloring methods, thin-layer chromatography, NIRS, enzyme assays or microbiological assays. These analytical methods are compiled in: Patek et al. (1994) Appl. Environ. Microbiol. 60: 133-140; Malakhova et al. (1996) Biotekhnologiya 11 27-32; and Schmidt et al. (1998) Bioprocess Engineer. 19: 67-70. Ulmann's Encyclopedia of Industrial Chemistry (1996) Vol. A27, VCH: Weinheim, pp. 89-90, pp. 521-540, pp. 540-547, pp. 559-566, 575-581 and pp. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Vol. 17.

Equivalents

The skilled worker knows, or can identify by using simply routine methods, a large number of equivalents of the specific embodiments of the invention. These equivalents are intended to be included in the patent claims below.

The information in Table 1 is to be understood as follows:

In column 1, “DNA ID”, the relevant number refers in each case to the SEQ ID NO of the enclosed sequence listing. Consequently, “5” in column “DNA ID” is a reference to SEQ ID NO:5.

In column 2, “AA ID”, the relevant number refers in each case to the SEQ ID NO of the enclosed sequence listing. Consequently, “6” in column “AA ID” is a reference to SEQ ID NO:6.

In column 3, “Identification”, an unambiguous internal name for each sequence is listed.

In column 4, “AA pos”, the relevant number refers in each case to the amino acid position of the polypeptide sequence “AA ID” in the same row. Consequently, “26” in column “AA pos” is amino acid position 26 of the polypeptide sequence indicated accordingly. Position counting starts at the N terminus with +1.

In column 5, “AA wild type”, the relevant letter refers in each case to the amino acid, displayed in the one-letter code, at the position in the corresponding wild-type strain, which is indicated in column 4.

In column 6, “AA mutant”, the relevant letter refers in each case to the amino acid, displayed in the one-letter code, at the position in the corresponding mutant strain, which is indicated in column 4.

In column 7, “Function”, the physiological function of the corresponding polypeptide sequence is listed.

One-letter code of the proteinogenic amino acids:

A Alanine

C Cysteine

D Aspartic acid

E Glutamic acid

F Phenylalanine

G Glycine

H Histidine

I Isoleucine

K Lysine

L Leucine

M Methionine

N Asparagine

P Proline

Q Glutamine

R Arginine

S Serine

T Threonine

V Valine

W Tryptophan

Y Tyrosine

TABLE 1
Genes coding for DNA replication and pathogenesis proteins

| | | | AA | AA | AS | |
|---|---|---|---|---|---|---|
| DNA ID: | AA ID: | Identification: | Pos: | wild type | mutant | Function: |
| 1 | 2 | RXA00050 | 66 | A | V | ATP-DEPENDENT RNA HELICASE DEAD |
| | | | 596 | G | S | ATP-DEPENDENT RNA HELICASE DEAD |
| 3 | 4 | RXA00061 | 754 | S | N | DNA POLYMERASE I (EC 2.7.7.7) |
| 5 | 6 | RXA00157 | 90 | V | I | INVASIN 1 |
| | | | 159 | G | R | INVASIN 1 |
| | | | 449 | G | D | INVASIN 1 |
| | | | 454 | S | F | INVASIN 1 |
| | | | 590 | S | F | INVASIN 1 |
| 7 | 8 | RXA00208 | 45 | G | E | VULNIBACTIN UTILIZATION PROTEIN VIUB |
| | | | 61 | D | N | VULNIBACTIN UTILIZATION PROTEIN VIUB |
| | | | 76 | P | L | VULNIBACTIN UTILIZATION PROTEIN VIUB |
| 9 | 10 | RXA00313 | 69 | E | K | 23S RRNA METHYLTRANSFERASE (EC 2.1.1.—) |
| 11 | 12 | RXA00341 | 254 | G | D | EXORIBONUCLEASE II (EC 3.1.13.1) |
| | | | 414 | D | G | EXORIBONUCLEASE II (EC 3.1.13.1) |
| 13 | 14 | RXA00407 | 187 | D | H | DNA POLYMERASE III, ALPHA CHAIN (EC 2.7.7.7) |
| 15 | 16 | RXA00694 | 34 | A | T | SSU ribosomal protein S8P |
| 17 | 18 | RXA00696 | 66 | T | I | LSU ribosomal protein L18P |
| 19 | 20 | RXA00807 | 62 | G | E | DNA POLYMERASE III, DELTA′ SUBUNIT (EC 2.7.7.7) |
| | | | 241 | R | C | DNA POLYMERASE III, DELTA′ SUBUNIT (EC 2.7.7.7) |
| 21 | 22 | RXA01238 | 156 | S | N | DNA POLYMERASE III, ALPHA CHAIN (EC 2.7.7.7) |
| 23 | 24 | RXA01280 | 43 | K | R | SSU ribosomal protein S12P |
| 25 | 26 | RXA01305 | 400 | L | F | ADHESIN AIDA-I |
| | | | 545 | S | F | ADHESIN AIDA-I |
| 27 | 28 | RXA01343 | 11 | A | V | LSU ribosomal protein L1P |
| 29 | 30 | RXA01480 | 297 | G | D | DNA PRIMASE (EC 2.7.7.—) |
| 31 | 32 | RXA01563 | 117 | P | S | RIBONUCLEASE D (EC 3.1.26.3) |
| | | | 375 | R | Q | RIBONUCLEASE D (EC 3.1.26.3) |
| 33 | 34 | RXA01581 | 274 | S | F | 23S RRNA METHYLTRANSFERASE (EC 2.1.1.—) |
| 35 | 36 | RXA01661 | 64 | G | D | RRNA METHYLTRANSFERASE (EC 2.1.1.—) |
| 37 | 38 | RXA01683 | 130 | R | C | DNA GYRASE SUBUNIT A (EC 5.99.1.3) |
| | | | 584 | S | F | DNA GYRASE SUBUNIT A (EC 5.99.1.3) |
| 39 | 40 | RXA01718 | 284 | P | S | CBS domain containing protein |
| 41 | 42 | RXA01736 | 236 | P | S | PUTATIVE ATP-DEPENDENT DNA HELICASE |
| 43 | 44 | RXA01770 | 243 | D | N | DNA HELICASE II (EC 3.6.1.—) |
| 45 | 46 | RXA01772 | 502 | S | F | Superfamily II DNA and RNA helicase |
| 47 | 48 | RXA01949 | 97 | S | N | LSU ribosomal protein L23P |
| 49 | 50 | RXA01966 | 46 | A | V | OLIGORIBONUCLEASE (EC 3.1.—.—) |
| 51 | 52 | RXA02070 | 150 | A | T | MRP PROTEIN HOMOLOG |
| 53 | 54 | RXA02082 | 726 | A | V | CHROMOSOME SEGREGATION PROTEIN SMC2 |
| | | | 781 | L | F | CHROMOSOME SEGREGATION PROTEIN SMC2 |
| | | | 905 | A | T | CHROMOSOME SEGREGATION PROTEIN SMC2 |
| 55 | 56 | RXA02145 | 321 | P | L | MENAQUINOL-CYTOCHROME C REDUCTASE CYTOCHROME B SUBUNIT |
| 57 | 58 | RXA02293 | 349 | A | V | ATP-DEPENDENT HELICASE HRPB |
| 59 | 60 | RXA02357 | 896 | G | E | PROBABLE ATP-DEPENDENT HELICASE LHR (EC 3.6.1.—) |
| | | | 1414 | T | I | PROBABLE ATP-DEPENDENT HELICASE LHR (EC 3.6.1.—) |
| 61 | 62 | RXA02363 | 728 | V | M | ATP-dependent helicase |
| 63 | 64 | RXA02533 | 142 | R | C | TRANSCRIPTIONAL REGULATORY PROTEIN |
| 65 | 66 | RXA02657 | 840 | H | Y | PROBABLE DNA POLYMERASE III, ALPHA CHAIN (EC 2.7.7.7) |
| | | | 1161 | D | N | PROBABLE DNA POLYMERASE III, ALPHA CHAIN (EC 2.7.7.7) |
| 67 | 68 | RXA03166 | 1183 | P | L | ATP-DEPENDENT HELICASE HRPA |
| 69 | 70 | RXA03266 | 103 | T | I | DNA POLYMERASE III, BETA CHAIN (EC 2.7.7.7) |
| | | | 364 | P | L | DNA POLYMERASE III, BETA CHAIN (EC 2.7.7.7) |
| 71 | 72 | RXA03332 | 52 | G | E | phage Hau3 resistance protein |
| 73 | 74 | RXA03341 | 153 | E | K | DNA-BINDING PROTEIN |
| 75 | 76 | RXA03590 | 398 | P | S | virulence-associated protein E |
| 77 | 78 | RXA03607 | 176 | R | K | Hypothetical phage protein |
| 79 | 80 | RXA03743 | 157 | S | F | DNA TOPOISOMERASE I (EC 5.99.1.2) |
| 81 | 82 | RXA03903 | 811 | E | K | DNA TOPOISOMERASE I (EC 5.99.1.2) |
| 83 | 84 | RXA04194 | 922 | R | C | ATP-DEPENDENT DNA HELICASE |
| 85 | 86 | RXA07017 | 574 | P | S | DNA POLYMERASE III SUBUNITS GAMMA AND TAU (EC 2.7.7.7) |
| 87 | 88 | RXA07018 | 1245 | L | F | DNA-DIRECTED RNA POLYMERASE BETA′ CHAIN (EC 2.7.7.6) |
| 89 | 90 | RXA07019 | 55 | G | S | LSU ribosomal protein L2P |
| 91 | 92 | RXA07020 | 314 | R | S | PHENYLALANYL-TRNA SYNTHETASE ALPHA CHAIN (EC 6.1.1.20) |
| 93 | 94 | RXA07027 | 256 | G | D | PHAGE INFECTION PROTEIN |
| 95 | 96 | RXA07028 | 27 | E | K | TYPE II RESTRICTION-MODIFICATION SYSTEM RESTRICTION SUBUNIT |
| | | | 310 | A | V | TYPE II RESTRICTION-MODIFICATION SYSTEM RESTRICTION SUBUNIT |

We claim:

1. An isolated nucleic acid molecule, coding for a polypeptide having the amino acid sequence referred to in each case in table 1/column 2, wherein the nucleic acid molecule in the amino acid position indicated in table 1/column 4 encodes a proteinogenic amino acid different from the particular amino acid indicated in table 1/column 5 in the same row.

2. An isolated nucleic acid molecule as claimed in claim 1, wherein the nucleic acid molecule in the amino acid position indicated in table 1/column 4 encodes the amino acid indicated in table 1/column 6 in the same row.

3. A vector, which comprises at least one nucleic acid sequence as claimed in claim 1.

4. A host cell, which is transfected with at least one vector as claimed in claim 3.

5. A host cell as claimed in claim 4, wherein expression of said nucleic acid molecule modulates the production of a fine chemical from said cell.

6. A method for preparing a fine chemical, which comprises culturing a cell which has been transfected with at least one vector as claimed in claim 3 so that the fine chemical is produced.

7. A method as claimed in claim 6, wherein the fine chemical is an amino acid.

8. A method as claimed in claim 7, wherein said amino acid is lysine.
